SEQUENCE LISTING (19 sequences)

SEQ ID NO: 1 - protein, 330 aa, Homo sapiens [sequence omitted]

SEQ ID NO: 2 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 3 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 7 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 8 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 9 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 10 - protein, 126 aa, Homo sapiens
QAQVVESGGGVVQSGRSLRLSCAASGFAFSSYGMHWVRQAPGKGLEWVAVIWYDGSNKYYADSVRGRFTISRDNSENTLYLQMNSLRAEDTAVYYCARDHYGSGVHHYFYYGLDVWGQGTTVTVSS

SEQ ID NO: 11 - protein, 456 aa, Homo sapiens
QAQVVESGGGVVQSGRSLRLSCAASGFAFSSYGMHWVRQAPGKGLEWVAVIWYDGSNKYYADSVRGRFTISRDNSENTLYLQMNSLRAEDTAVYYCARDHYGSGVHHYFYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 456 aa, synthetic construct
QAQVVESGGGVVQSGRSLRLSCAASGFAFSSYGMHWVRQAPGKGLEWVAVIWYDGSNKYYADSVRGRFTISRDNSENTLYLQMNSLRAEDTAVYYCARDHYGSGVHHYFYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 14 - protein, 456 aa, synthetic construct
QAQVVESGGGVVQSGRSLRLSCAASGFAFSSYGMHWVRQAPGKGLEWVAVIWYDGSNKYYADSVRGRFTISRDNSENTLYLQMNSLRAEDTAVYYCARDHYGSGVHHYFYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 453 aa, Homo sapiens
QAQVVESGGGVVQSGRSLRLSCAASGFAFSSYGMHWVRQAPGKGLEWVAVIWYDGSNKYYADSVRGRFTISRDNSENTLYLQMNSLRAEDTAVYYCARDHYGSGVHHYFYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 453 aa, synthetic construct
QAQVVESGGGVVQSGRSLRLSCAASGFAFSSYGMHWVRQAPGKGLEWVAVIWYDGSNKYYADSVRGRFTISRDNSENTLYLQMNSLRAEDTAVYYCARDHYGSGVHHYFYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 453 aa, synthetic construct
QAQVVESGGGVVQSGRSLRLSCAASGFAFSSYGMHWVRQAPGKGLEWVAVIWYDGSNKYYADSVRGRFTISRDNSENTLYLQMNSLRAEDTAVYYCARDHYGSGVHHYFYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 452 aa, Homo sapiens
QAQVVESGGGVVQSGRSLRLSCAASGFAFSSYGMHWVRQAPGKGLEWVAVIWYDGSNKYYADSVRGRFTISRDNSENTLYLQMNSLRAEDTAVYYCARDHYGSGVHHYFYYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 19 - protein, 452 aa, synthetic construct
QAQVVESGGGVVQSGRSLRLSCAASGFAFSSYGMHWVRQAPGKGLEWVAVIWYDGSNKYYADSVRGRFTISRDNSENTLYLQMNSLRAEDTAVYYCARDHYGSGVHHYFYYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2026/0234271 A1
(43) Pub. Date: Aug. 13, 2026

(54) COMPOSITIONS AND USES THEREOF

(71) Applicant: BEECH BIOTECH SA, Lausanne (CH)

(72) Inventor: Ian COTTINGHAM, Lausanne (CH)

(21) Appl. No.: 19/153,446

(22) PCT Filed: Feb. 2, 2024
(86) PCT No.: PCT/EP2024/052607

§ 371 Date: Aug. 4, 2025

(30) Foreign Application Priority Data
Feb. 3, 2023 (GB) 2301536.5

(51) Int. Cl. C07K 16/28; A61K 39/00

(52) U.S. Cl. CPC C07K 16/2863; A61K 2039/505; C07K 2317/71; C07K 2317/92; C07K 2317/94

(57) ABSTRACT

Molecules, in particular polypeptides, including imnmunoglobulins (e.g., antibodies), comprising mutations that result in reduced maternofetal transfer, as well as nucleic acids encoding such polypeptides, therapeutic and diagnostic compositions, formulations, kits, and methods of making and using the molecules, including methods of treatment.

### FIELD

The present disclosure relates to molecules, in particular polypeptides, including but not limited to immunoglobulins (e.g., antibodies), comprising a variant IgG Fc domain comprising mutations that result in reduced maternofetal transfer. The disclosure also comprises nucleic acids encoding such polypeptides, expression vectors, host cells, and methods of making and using them, including therapeutic and diagnostic compositions, formulations, and kits.

### BACKGROUND

Drugs administered to the mother can be dangerous for the fetus thus making the development of new therapeutics very challenging. Animal models that reflect the same physiology and pathology as human pregnancy are very limited and therapeutic effects in models do not well reflect those in human. The maternal metabolism can change during different stages of pregnancy, and in pathological conditions, meaning that the exposure to the fetus is variable and difficult to define. The susceptibility of the fetus also changes during the progression of development and growth. Furthermore, the means to monitor the safety of the fetus during pregnancy are very limited and drug-related adverse effects cannot easily be detected or characterised. Taken together these limitations and safety concerns mean that very few drugs are approved for use during pregnancy, very few are in development and the prospects for new treatments are severely limited.

As such, there is an unmet need for a new class of therapeutic drugs capable of addressing the medical conditions of pregnant women (for instance, pre-existing auto-immune disorders or pregnancy-related hypertension or pre-eclampsia/eclampsia) while still remaining safe for the fetus.

This important problem is solved by the present invention by providing polypeptides, in particular antibodies, which are not transported across the placenta thus allowing the development of new drugs which can harness the broad therapeutic applicability and specificity of antibodies without concern for the safety of the fetus.

Purposely reducing the placental transfer ability of a polypeptide to solve the above-mentioned problem has never been attempted in the past. Placental transfer has been studied extensively (as you will see below) but always in an attempt to facilitate or increase placental transfer rather than inhibiting it.

The placenta functions to exchange all that the early embryo and developing fetus requires for life and to remove the waste products from fetal metabolism. It is selective in allowing the exchange of dissolved gasses, small molecules and nutrients but provides an almost complete barrier to larger molecules and proteins thus preventing the mixture of maternal and fetal blood components.

In women, maternal antibodies of the IgG sub-class can cross the placenta in increasing amounts from around week 20 of pregnancy. This permits the transfer of maternal antibodies which provide a level of immunity to the fetus until its own immune system can develop. The efficiency of antibody transfer depends on the IgG subclass, with IgG-1 most efficiently transferred, and relies on an active process since administered therapeutic antibodies can be measured at higher concentration (1.5 to 4 fold higher) in the fetal circulation compared to the mother (Pham-Huy, A., et al, From mother to baby: antenatal exposure to monoclonal antibody biologics. Expert Review of Clinical Immunology. Taylor and Francis Ltd; 2019, 15: 221-9).

Transport of antibodies from mother to fetus is not always beneficial. The presence of auto-antibodies to Ro (SS-A) and La (SS-B) in pregnant women with or without a full-blown autoimmune disease conveys a substantially increased risk of the neonatal lupus syndrome. Similarly, prospective studies of anticardiolipin autoantibodies in pregnant women have shown a significantly increased frequency of mid-trimester fetal loss in women with high levels of such autoantibodies (Elkon K., et al, Nature and functions of autoantibodies. Nat. Clin. Pract. Rheumatol. 2008, 9:491-8).

The majority of therapeutic antibodies in clinical use are of the IgG class and there are many reports of placental transfer (Pham-Huy, K. A., et al, The use and impact of monoclonal antibody biologics during pregnancy. CMAJ 2021, 193: 1129-1136). Adulimumab, for example, is found at levels up to 1.5 times higher in the neonate compared to the maternal circulation and the drug can persist for 3-5 months in the infant circulation. The transfer of therapeutic antibodies across the placenta is a real medical concern because of known dangers for the fetus or a lack of information about the risk to the fetus. An example of known risk is bevacizumab which inhibits angiogenesis and causes fetal defects in animals and according to the drug label is not recommended during pregnancy. The risk of a low incidence but serious adverse effect for the fetus following treatment of pregnant women is very difficult to assess and today only products that have been in long-term use with large registry databases can be assessed for fetal safety. The risk of adverse effects due to fetal exposure limits the use of therapeutic antibodies in pregnant women meaning that treatment for an ongoing condition of the mother is normally halted around the end of the first trimester, after which significant maternal antibody transfer occurs, unless the benefit to the mother outweighs the risk to the fetus. Currently developed therapeutic antibodies can thus only be administered to pregnant women with acceptance of fetal risk which severely limits the available antibody treatments for pregnant women.

Natural IgG antibodies are tetrameric proteins comprising two identical heavy chains and two identical light chains with the variable regions of each heavy chain/light chain pair juxtaposed to form two identical antigen binding sites. The carboxy-terminal portion of the heavy chain regions, after the so-called ‘hinge region’, which are not paired with light chains and are linked to each other by disulphide bridges comprise the so-called Fc region. The Fc region contains binding sites which interact with a range of different Fcγ receptors and the FcRn receptor as described below. Transfer of IgG across the maternal syncytiotrophoblast membrane and the fetal placental endothelial cell layer is a complex, incompletely understood process and the role of different Fc receptors is debated.

The FcRn receptor is known to play a major role in placental transfer of IgG. This was demonstrated, although not with the purpose of providing a polypeptide according to the invention, by introducing single mutations in the antibody Fc region to interfere with binding to the FcRn receptor:
Disruption of Fc—FcRn binding by substitution of histidine for alanine at amino acid 435 was shown to reduce materno-fetal transfer in both pregnant mice and across ex-vivo human placenta by as much as 90% (Firan, M., et al, The MHC class I-related receptor, FcRn, plays an essential role in the maternofetal transfer of gamma-globulin in humans. Int. Immunol. 2001, 13: 993-1002).
Single FcRn mutations I253A, H310A or H435A reduce maternofetal transfer in pregnant mice by 80 to 90% but were not tested for the effects on maternofetal transfer in combination (see U.S. Pat. No. 6,277,375 B1).
A modified version of bevacizumab with the H435A Fc mutation was tested in a rat model and found to block maternofetal transfer by no more than 90% (Thorn, M., et al, Embryo-fetal transfer of bevacizumab (Avastin) in the rat over the course of gestation and the impact of neonatal Fc receptor (FcRn) binding. Birth Defects Res B Dev Reprod. Toxicol. 2012, 95: 363-75)

Although FcRn plays a major role in IgG transfer there are no studies showing that the inhibition of maternofetal transfer following modifications to FcRn binding is reduced by more than 90%. This indicates that other receptors must be involved. Furthermore FcRn is not present on the fetal endothelium—which would be expected for a FcRn-only transport process—and maternofetal IgG selective transfer depending on glycosylation or antigen specificity has been reported—which cannot be explained by the selectivity of FcRn binding. Thus FcγR receptors IIa and IIIb have been reported to play a role in maternofetal transfer of antibodies (Jennewein, M. F., et al, Fc Glycan-Mediated Regulation of Placental Antibody Transfer. Cell. 2019, 178: 202-215). Based on the current state of research it cannot be excluded that other FcγR, in addition to IIa and IIIb, play a role in maternofetal transfer of IgG antibodies.

In the above cited prior-art, inhibition of maternofetal transfer by more than 90% has not been demonstrated and such level of inhibition would not be enough to provide a safe treatment for pregnant women.

### SUMMARY

Development of any therapeutic for the treatment of pregnant women, or women who may become pregnant, faces major medical concerns due to fetal exposure and the possible consequences for safety. This important problem is solved herein by the design of antibodies which are not transported across the placenta allowing the development of new drugs which can harness the broad therapeutic applicability, specificity, and known manufacturing technologies of antibodies without concern for the safety of the fetus. Such ‘mother-only’ antibodies enable the creation of a brand-new class of therapeutics to address the unmet medical needs of pregnant women such as hypertension-related conditions and pre-eclampsia/eclampsia, and other disorders (diseases) of pregnancy.

It is also possible to develop antibodies, and other IgG Fc containing molecules as described herein, for conditions not related to pregnancy which can still be used by women likely to become pregnant and during pregnancy.

It is therefore understood that the invention is not limited to any specific disease or treatment but provides a platform for the design of drugs, and drugs, with which pregnant women might be treated safely for a range of different disorders without risk of these harming the fetus or significantly reducing any risk to the fetus.

In one preferred aspect the invention relates to the treatment or prevention of a disorder in a pregnant woman using a therapeutic agent, the agent having an variant Fc domain as herein disclosed, such as a variant polypeptide, such as a variant antibody, wherein the therapeutic agent is suitable for treatment or prevention of the disorder, for example, is suitable for the treatment of a disorder of pregnancy, such as for treatment of pre-eclampsia or hypertension.

The present disclosure is directed to recombinant polypeptides comprising a variant Fc domain with amino acid substitutions resulting in a reduction of maternofetal transfer by more than 90%, preferably more than 95% and preferably by more than 96%, more than 97%, or more than 98%.

The polypeptide of the disclosure comprises a human variant IgG Fc domain comprising amino acid substitutions numbered according to the Kabat EU index numbering system, relative to a human parent Fc domain, which optionally is a wild type domain, wherein:
a) (i) positions 234 and 235 are each substituted with alanine or
   (ii) positions 234 and 235 are each substituted with alanine and position 331 is substituted with serine or
   (iii) position 234 is substituted with phenylalanine, position 235 is substituted with glutamic acid and position 331 is substituted with serine or
   (iv) position 328 is substituted with arginine and arginine is inserted after position 236; and
b) (i) position 253 is substituted with alanine or
   (ii) position 435 is substituted with alanine or
   (iii) positions 235, 310 and 435 are substituted with alanine or
   (iv) position 310 is substituted with alanine and position 435 is substituted with glutamine

In addition to a role in maternofetal transfer of antibodies, the FcRn receptor is important for maintaining the long half-life of IgG in the circulation. Blood proteins are subject to endocytosis by endothelial cells and directed to catabolic degradation. IgG, however, binds to the FcRn receptor in the low pH of the endosomes and is recycled to the endothelium and released in this neutral pH environment. Disabling the binding to FcRn prevents the operation of this rescue mechanism meaning the IgG half-life will be reduced, for example, from five days to two days as demonstrated in the mouse (see U.S. Pat. No. 6,277,375 Bi). The normal half-life for natural sequence antibodies in humans is about 10 to 20 days and is reduced from 2 to 10 fold by inhibition of recycling.

In the treatment of pregnant women with “mother-only” antibodies the shorter half-life is an advantage. The relatively short duration of pregnancy means a long-term treatment is not necessary and a therapeutic antibody can be given by infusion in the short term if high doses are of benefit. One major modality of therapeutic antibodies is to bind and remove soluble target proteins from the circulation with the example of sVEGFR-1 (Vascular endothelial growth factor receptor-1) during pregnancy. A short half-life means that such targets are bound and removed quickly from the maternal circulation and can even be titrated down in concentration with the antibody if, as for sVEGFR-1, the concentration can be conveniently measured. Infusion of an antibody with a shorter half-life is also a safety advantage since treatment can be interrupted if important safety issues arise and the antibody is quickly eliminated. A further advantage of a shorter half-life for the mother is that the therapeutic antibody does not persist after delivery when the treatment may no longer have utility for the mother or indeed be potentially harmful. Preferably a polypeptide, eg a therapeutic antibody, of the invention has a half-life which is reduced by a factor of at least 2 fold, at least 3 fold, at least 5 fold, at least 10 fold or more compared to the same molecule with the ‘parent’ Fc domain

Antibodies transferred from the mother to fetus, for example during therapeutic treatment of a pre-existing disease of the mother, can persist for many weeks indicating that the FcRn recycling mechanism responsible for the persistence of IgG in the circulation is active also in neonates. Conversely, removal of FcRn binding means that even if “mother-only” antibodies have a low level of placental transfer they will be quickly eliminated in the neonate and any possible risk will be further reduced.

Disabling of the FcγRs is also a desirable feature for treatments against targets that are soluble proteins but also have membrane equivalents in that immune effector dependent activities, such as antibody-directed cellular cytotoxicity, which could mediate unwanted and off-target side effects will be greatly reduced or abolished.

The present disclosure also provides an isolated nucleic acid comprising a sequence encoding the polypeptide of the disclosure. Also provided are compositions, expression vectors, and host cells which comprise a nucleic acid comprising a sequence encoding the polypeptide of the disclosure. The host cell can comprise an isolated nucleic acid comprising a sequence encoding the polypeptide of the disclosure, a composition comprising a nucleic acid comprising a sequence encoding the polypeptide of the disclosure, or an expression vector comprising a nucleic acid comprising a sequence encoding the polypeptide of the disclosure.

The present disclosure also provides a method of making a polypeptide of the disclosure comprising (a) culturing host cells comprising a nucleic acid comprising a sequence encoding the polypeptide of the disclosure; and, (b) isolating the polypeptide. The present disclosure also provides a composition comprising a polypeptide of the disclosure and a carrier.

The present disclosure also provides a conjugate comprising a polypeptide of the disclosure and a therapeutic moiety.

The present disclosure also provides a method of treating a mammal, preferably a human, preferably a woman, more preferably a pregnant woman comprising administering to a patient in need of treatment an effective amount of (a) a polypeptide of the disclosure, (b) an isolated nucleic acid comprising a sequence encoding the polypeptide of the disclosure, (c) a composition, expression vector, or host cell which comprises a nucleic acid comprising a sequence encoding the polypeptide of the disclosure, (d) a composition comprising a polypeptide of the disclosure and a carrier, or (e) a conjugate comprising a polypeptide of the disclosure and a therapeutic moiety.

The present disclosure also provides a method to reduce binding to at least one FcγR receptor and to FcRn in a parent polypeptide comprising an Fc domain comprising the steps of:
a) (i) positions 234 and 235 are each substituted with alanine or
   (ii) positions 234 and 235 are each substituted with alanine and position 331 is substituted with serine or
   (iii) position 234 is substituted with phenylalanine, position 235 is substituted with glutamic acid and position 331 is substituted with serine or
   (iv) position 328 is substituted with arginine and arginine is inserted after position 236; and
b) (i) position 253 is substituted with alanine or
   (ii) position 435 is substituted with alanine or
   (iii) positions 235, 310 and 435 are substituted with alanine or
   (iv) position 310 is substituted with alanine and position 435 is substituted with glutamine.

Other preferred features of the invention include:

An isolated polypeptide for use in medicine in a pregnant woman, wherein the polypeptide comprises a human variant IgG Fc domain having a mutation or combination of mutations with respect to the parent sequence which reduces binding of the Fc domain to any FcγR, and a mutation or combination of mutations with respect to the parent sequence which reduce binding of the Fc domain to FcRn, whereby maternofetal transfer is inhibited with respect to the parent sequence by at least 95% or more.

An isolated polypeptide for use in the treatment or prevention of a disorder of pregnancy in a pregnant woman, the polypeptide comprising a human variant IgG Fc domain having a mutation or combination of mutations with respect to the parent sequence which reduce binding of the Fc domain to any FcγR, and a mutation or combination of mutations with respect to the parent sequence which reduce binding of the Fc domain to FcRn, whereby maternofetal transfer is inhibited with respect to the parent sequence by at least 95% or more.

An isolated polypeptide for use as disclosed herein, or a method of treatment or nucleic acid or cell or vector for use, as disclosed herein, wherein the use is in the treatment or prevention of a disorder of pregnancy, for example wherein
(i) the disorder or disease is a hypertension-related condition or pre-eclampsia/eclampsia, or
(ii) wherein the disorder or disease is pre-eclampsia/eclampsia, and the polypeptide is an anti-VEGFR-1 antibody which inhibits binding of VEGF and/or PGF to sVEGFR-1 or
(iii) wherein the disorder or disease is pre-eclampsia/eclampsia, and the polypeptide comprises or consists of the polypeptide of SEQ ID NO 13, provided together with an antibody light chain of SEQ ID NO:12, in the form of an antibody, optionally in the form of a pharmaceutically acceptable composition in combination with a pharmaceutically acceptable excipient.

An antibody comprising the polypeptide of SEQ ID NO 13 together with an antibody light chain of SEQ ID NO:12, optionally formulated as a pharmaceutically acceptable composition with an excipient or carrier.

### FIGURES

FIG. 1 is a gel electrophoresis of purified antibodies WBP70323_1 (MOm301) and WBP70323_2 (MOm303)

FIG. 2 shows fold changes of total fluorescence signal of pregnant mice and foetuses compared to a PBS control. Panel A; pregnant mice prior to dissection of foetuses. Panel B; foetuses

FIG. 3 shows Plasma concentrations of BB301 and BB303 in pregnant mice and foetuses 24 hours after dosing. Plasma levels in the PBS controls were negative and are not shown.

### DETAILED DESCRIPTION

The present disclosure is directed to recombinant polypeptides comprising a variant Fc domain with amino acid substitutions resulting in reduced maternofetal transfer. The present disclosure relates in particular to polypeptides, more particularly immunoglobulins, comprising an IgG Fc domain (e.g., a human IgG Fc domain), or a fragment thereof (preferably an Fc or hinge-Fc domain) that contains one or more amino acid modifications relative to a parent IgG, which is optionally a wild type IgG sequence, and wherein such modifications greatly reduce both FcγR binding and FcRn binding.

In some aspects, the present disclosure particularly relates to the modification of human or humanized IgGs and other bioactive molecules containing FcRn-binding portions of human IgG Fc domains, which have particular use in therapy, prophylaxis and diagnosis. In some aspects, the polypeptides comprise an IgG Fc domain, or fragment thereof (preferably an Fc or hinge-Fc domain) comprising modifications inhibiting FcγR binding and FcRn binding.

### Definitions

It is to be noted that the term “a” or “an” entity refers to one or more of that entity; for example, “a polypeptide sequence,” is understood to represent one or more polypeptide sequences. As such, the terms “a” (or “an”), “one or more,” and “at least one” can be used interchangeably herein. Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their System International of Units (SI) accepted form. Numeric ranges are inclusive of the numbers defining the range. Unless otherwise indicated, amino acid sequences are written left to right in amino to carboxy orientation. The headings provided herein are not limitations of the various aspects, which can be had by reference to the specification as a whole. Accordingly, the terms defined immediately below are more fully defined by reference to the specification in its entirety.

It is understood that wherever aspects are described herein with the language “comprising,” otherwise analogous aspects described in terms of “consisting of” and/or “consisting essentially of” are also provided.

Amino acids can be referred to herein by either their name, their commonly known three letter symbols or by the one-letter symbols recommended by the IUPAC-IUB Biochemical Nomenclature Commission. Nucleotides, likewise, are referred to by their commonly accepted single-letter codes.

As used herein, the term “polypeptide” refers to a molecule composed of monomers (amino acids) linearly linked by amide bonds (also known as peptide bonds). The term “polypeptide” refers to any chain or chains of two or more amino acids, and does not refer to a specific length of the product. As used herein the term “protein” is intended to encompass a molecule comprised of one or more polypeptides, which can in some instances be associated by bonds other than amide bonds. On the other hand, a protein can also be a single polypeptide chain. In this latter instance the single polypeptide chain can in some instances comprise two or more polypeptide subunits fused together to form a protein. The terms “polypeptide” and “protein” also refer to the products of post-expression modifications, including without limitation glycosylation, acetylation, phosphorylation, amidation, derivatization by known protecting/blocking groups, proteolytic cleavage, or modification by non-naturally occurring amino acids. A polypeptide or protein can be derived from a natural biological source or produced by recombinant technology, but is not necessarily translated from a designated nucleic acid sequence. It can be generated in any manner, including by chemical synthesis.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells or compositions include those which have been purified to a degree that they are no longer in a form in which they are found in nature. In some aspects, an antibody, polynucleotide, vector, cell, or composition which is isolated is substantially pure.

A reference to an isolated polypeptide comprising a human variant IgG Fc domain herein can be a reference to a single polypeptide or, more preferred, refers to a combination of two separate polypeptides that together form an Fc domain e.g. via one or more disulphide bonds, which might also be described as a protein comprising a human variant IgG Fc domain herein.

A “recombinant” polypeptide or protein refers to a polypeptide or protein produced via recombinant DNA technology. Recombinantly produced polypeptides and proteins expressed in host cells are considered isolated for the purpose of the present disclosure, as are native or recombinant polypeptides which have been separated, fractionated, or partially or substantially purified by any suitable technique.

Also included in the present disclosure are fragments, variants, or derivatives of polypeptides, and any combination thereof. The term “fragment” when referring to polypeptides and proteins of the present disclosure include any polypeptides or proteins which retain at least some of the properties of the reference polypeptide or protein. Fragments of polypeptides include proteolytic fragments, as well as deletion fragments.

The term “variant” as used herein refers to a polypeptide sequence that differs from that of its parent polypeptide sequence by virtue of at least one amino acid modification. The parent polypeptide can be a naturally occurring polypeptide including known allotypes, i.e., a “wild-type” (“wt”) polypeptide, or can be a modified version of a wild-type polypeptide that does not already contain all of the Fc domain amino acid substitutions disclosed herein, and into which any one of the substitutions disclosed herein can be introduced, resulting in a change in Fc domain sequence.

Examples of modifications to natural wild type IgG sequences which are incorporated in currently approved antibodies include but are not limited to: IgG1(N297A), IgG1(N297G), IgG1(L234F/L235E/P331S), IgG1(L234A/L235A), IgG1(L235V/F243L/R292P/Y300L/P396L), IgG1(L234F/L235E/P331S), IgG1(L234A/L235A/P329G), IgG1(S354C/T366W), IgG1(Y349C/T366S/L368A/Y407V), IgG1(M252Y/S254T/T256E), IgG1/2(S239D/I332E), IgG2(C131S/R133K/C219S), IgG2(H268Q/R355Q/Q419E/N434A), IgG2/4(M428L/N434S), IgG4(S228P), IgG4(S228P/F234A/L235A), IgG4(S228P/L235E), IgG4(S241P/F234A/L235A), IgG4(F405L/R409K). Thus an IgG sequence which is a natural wild type allotype with any of these modifications, or combination of modifications, is a ‘parent’ polypeptide or Fc domain, as defined herein, and the parent amino acid sequence can be then modified to include the Fc domain amino acid substitutions as disclosed herein. Known human allotype sequences are listed in a number of databases, for example ImMunoGeneTics (www.imgt.org).

The term variant polypeptide can refer to the polypeptide itself, a composition comprising the polypeptide, or the amino sequence that encodes it. Preferably, the variant polypeptide (e.g., a polypeptide comprising a variant IgG Fc domain) has at least one amino acid modification compared to the parent polypeptide, e.g., from about one to about ten amino acid modifications, and preferably from about one to about six amino acid modifications compared to the parent polypeptide. The variant polypeptide sequence herein will generally possess at least about 90% sequence identity with a parent polypeptide sequence, and most generally at least about 95% sequence identity, for example when considered over the region of the whole antibody chain.

Variants of polypeptides or proteins of the present disclosure include fragments as described above, and also polypeptides or proteins with altered amino acid sequences due to amino acid substitutions, deletions, or insertions. Variants can be naturally or non-naturally occurring. Non-naturally occurring variants can be produced using mutagenesis techniques known in the art. Variant polypeptides can comprise conservative or non-conservative amino acid substitutions, deletions or additions.

The term “derivatives” as applied to polypeptides or proteins refers to polypeptides or proteins which have been altered so as to exhibit additional features not found on the native polypeptide or protein. An example of a “derivative” of a variant Fc domain is a fusion or a conjugate with a second polypeptide or another molecule (e.g., a polymer, a chromophore, or a fluorophore) or a chelating chemical structure capable of binding an atom (e.g., a radioisotope).

The terms “polynucleotide” or “nucleotide” as used herein are intended to encompass a singular nucleic acid as well as plural nucleic acids, and refers to an isolated nucleic acid molecule or construct, e.g., messenger RNA (mRNA) or plasmid DNA (pDNA). In certain aspects, a polynucleotide comprises a conventional phosphodiester bond or a nonconventional bond (e.g., an amide bond, such as found in peptide nucleic acids (PNA)).

The term “nucleic acid” refers to any one or more nucleic acid segments, e.g., DNA or RNA fragments, present in a polynucleotide. When applied to a nucleic acid or polynucleotide, the term “isolated” refers to a nucleic acid molecule, DNA or RNA, which has been removed from its native environment, for example, a recombinant polynucleotide encoding a polypeptide comprising a variant Fc domain contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

As used herein, the term “host cell” refers to a cell or a population of cells harboring or capable of harboring a recombinant nucleic acid. Host cells can be a prokaryotic cells (e.g., E. coli), or alternatively, the host cells can be eukaryotic, for example, fungal cells (e.g., yeast cells such as Saccharomyces cerivisiae, Pichia pastoris, or Schizosaccharomyces pombe), and various animal cells, such as insect cells (e.g., Sf-9) or mammalian cells (e.g., HEK293F, CHO, COS-7, NIH-3T3, PERC6).

The present disclosure also encompasses polypeptides comprising a variant IgG Fc domain comprising one or more conservative amino acid substitutions. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

The term “percent sequence identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof, based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc. The terms “antibody” or “immunoglobulin,” as used interchangeably herein, include whole antibodies and any antigen binding fragment or single chains thereof.

The term “IgG” as used herein refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, and IgG3.

The term “antigen binding fragment” refers to a portion of an intact antibody and refers to the antigenic determining variable regions of an intact antibody. It is known in the art that the antigen binding function of an antibody can be performed by fragments of a full-length antibody. Examples of antibody fragments include, but are not limited to Fab, Fab′, F(ab′)2, and Fv fragments, linear antibodies, single chain antibodies, and multispecific antibodies formed from antibody fragments.

The term “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, by hybridoma, phage selection, recombinant expression, and transgenic animals.

The term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides. The term “humanized antibody” refers to an antibody derived from a non-human (e.g., murine) immunoglobulin, which has been engineered to contain minimal non-human (e.g., murine) sequences.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc) with the desired specificity, affinity, and capability while the constant regions are homologous to the sequences in antibodies derived from another (usually human) to avoid eliciting an immune response in that species.

IgG immunoglobulins naturally comprise a pair of ‘heavy chain’ polypeptides, containing around 450 amino acids in IgG1, and a pair of ‘light chains’, containing around 110 amino acids in IgG1, which are bound in amino-terminal to amino-terminal orientation with each heavy chain, a structure represented by a cartoon ‘Y’ shape. The arms of the Y carry the ‘complementarity-determining regions’, which are binding sites for molecular targets of the antibody formed between the ‘so-called’ variable regions of each pair of heavy and light chains. These ‘variable’ regions of the protein can have many thousands of different sequences depending on the structure of the binding sites and the nature of the target and normally determine the binding properties of the antibody. The carboxy-terminal regions of the two heavy chains are linked to form the trunk of the Y. Treatment of a whole antibody with the enzyme ‘papain’ cleaves at the junction of the Y and releases the two arms, known as Fab fragments and carrying the target binding regions, and a single trunk fragment which, due to being readily crystallisable, is known as the Fc (fragment crystallisable) region. The amino-terminal regions of the light and heavy chains thus contain variable regions which determine ‘complementarity regions’ designated VL and VH respectively. A light chain also comprises a so-called ‘constant’ region, CL, and heavy chains comprise three ‘constant’ regions known as CHi, CH2 and CH3 in the human IgG nomenclature. In this context ‘constant’ is to distinguish from the highly variable complementarity-determining regions (CDR) sequences and these ‘constant’ sequences are subject to natural variations, allotypes, due to population genetic polymorphisms. In the intact antibody the constant region of the light chain, CL, and the first constant region of the heavy chain for the lower part of the arms of the ‘Y’ and the ‘hinge region’ of an antibody is normally between CH1 and CH2.

The treatment of an intact antibody with the enzyme papain cleaves both heavy chains between CH1 and CH2 constant domains so that the resulting Fc fragment comprises a dimer of two heavy chains with some of the hinge region sequence and the full constant domains CH2 and CH3. Sequences for Human Fc variants are presented herein as the entire heavy chain constant region including CH1, the hinge region and regions CH2 and CH3 and normally start with the sequence alanine-serine-threonine-lysine-glycine. However it will be clear to a skilled person that ‘Fc domain’ normally refers to a dimer comprising the CH2 and CH3 regions from two individual heavy chains and that the full sequence of the constant heavy chain region is provided for clarity and consistency across antibody types: IgG 1 to IgG4 for example.

The term “Kabat EU index numbering system” refers to the numbering system of the human IgG1 EU antibody described in Kabat et al., Sequences of Immunological Interest, 5th Ed. Public Health Service, National Institutes of Health, Bethesda, Md. (1991). Thus the Kabat EU index numbering system (see pages 661-723) is used for the constant heavy chain domains (CHI, Hinge, CH2 and CH3). For example, both “L234” and “EU L234” refer to the amino acid leucine at position 234 according to the Kabat EU index numbering system.

The terms “Fc domain” and “IgG Fc domain” as used herein refer to the Fc region of an immunoglobulin, e.g., an IgG molecule, which comprises the C-terminal half of two heavy chains of an IgG molecule that are linked by disulfide bonds. It has no antigen binding activity but contains the carbohydrate moiety and binding sites for complement and Fc receptors, including the FcRn receptor. For example, the Fc region contains a part of the hinge region plus the entire second constant domain CH2 (residues 231-340 of human IgG1, according to the Kabat EU index numbering system and the third constant domain CH3 (residues 341-447). Fc can refer to this region in isolation, or this region in the context of an antibody, antibody fragment, or Fc fusion protein.

Despite being designated ‘constant’ regions in the natural sequences of antibodies the CH 1-3 regions have small differences in sequence between individuals and between populations. Such differences, known as allotypes and polymorphisms, often occur as single amino-acid changes and have been observed at a number of positions in Fc domains both between classes, meaning IgG 1, 2, 3 and 4, and within classes. For example natural allotypes (variants) of IgG1 include G1m3, G1m17,1 or G1m7,1,2 allotypes or G1m(f), G1m(z,a), G1m(z,a,x) which differ by one to ten or more amino acid changes in the heavy chain constant region. (DeTaeye, S. W., et al, FcγR Binding and ADCC Activity of Human IgG Allotypes, Frontiers in Immunology 2020, 11: 1-16). A number of databases provide examples of known polymorphisms such as the international lniMunoGeneTics infornation system (www.imgt.org). Thus, a “wild type IgG Fc domain” or “wt IgG Fc domain” refers to any naturally occurring IgG Fc region and all polymorphisms, allotypes and alleles.

Sequences of the constant regions of the heavy chains of human IgG1, IgG2, IgG3 and IgG4 can be found in a number of sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG4_HUMAN), respectively. The sequences for the constant regions of heavy chains for these specific alleles of IgG1-4 are presented, starting at position 119 according to the Kabat EU index numbering system: IgG1 (SEQ ID NO:1), IgG2 (SEQ ID NO:2), IgG3 (SEQ ID NO:3) and IgG4 (SEQ ID NO:4).

IgG light chains can take the form of either so-called Kappa sequences or Lamba sequences. Light chains presented in the application include either form, and include polymorphisms in the constant light chain region.

The terms “variant IgG Fc domain” and “IgG Fc variant domain” as used herein refers to an IgG Fc domain comprising one or more amino acid substitutions, deletions, insertions or modifications introduced at any position within the Fc domain. In certain aspects a variant IgG Fc domain comprises one or more amino acid substitutions resulting in decreased or ablated binding affinity for an FcγR and FcRn as compared to the parent Fc domain (which may be a wild type domain) not comprising the one or more amino acid substitutions.

The term “Fc fusion” as used herein refers to a protein in which one or more polypeptides or small molecules are operably linked to an Fc domain or a variant or derivative thereof. An Fc fusion combines the Fc region of an immunoglobulin with a fusion partner, which in general can be any protein or small molecule. The role of the non-Fc part of an Fc fusion, i.e., the fusion partner, can be to mediate target binding, and thus it can be functionally analogous to the variable regions of an antibody.

The term “parent” polypeptide as used herein refers to a polypeptide (e.g., a parent Fc domain, or a polypeptide comprising an Fc domain such as antibody or Fc fusion) that is subsequently modified to generate a variant (e.g., a variant Fc domain, or a variant polypeptide comprising an Fc domain such as a variant antibody or a variant Fc fusion). The parent polypeptide can be a naturally occurring polypeptide (e.g., a wild type Fc domain), or a variant or engineered version of a naturally occurring polypeptide. The term parent polypeptide can refer to the polypeptide itself, compositions that comprise the parent polypeptide, or the amino acid sequence that encodes it. Accordingly, by “parent Fc domain” as used herein is meant a Fc domain that is modified to generate a variant, and by “parent antibody” as used herein is meant an antibody that is modified to generate a variant antibody comprising an IgG variant Fc domain.

An “Fc variant” comprises an Fc domain and can exist alone or in the context of an antibody, Fc fusion, isolated Fc, Fc fragment, or other polypeptide. Fc variants can refer to the Fc polypeptide itself, compositions comprising the Fc variant polypeptide, or the amino acid sequence that encodes it. The variant IgG Fc domains described herein are defined according to the amino acid modifications that compose them. For all amino acid positions discussed herein, numbering is always according to the Kabat EU index numbering system. Thus, for example, L234A is an Fc variant with the leucine (L) at EU position 234 substituted with alanine (A) relative to the parent Fc domain. Likewise, e.g., L234A/L235A/L328R defines a variant Fc variant with substitutions at EU positions 234 (L to A), 235 (L to A), and 328 (L to R) relative to the parent Fc domain. The terms “Fc gamma receptor” or “FcγR” as used herein refer to any member of the family of proteins that bind the IgG antibody Fc region and are encoded by the FcγR genes. In humans this family includes but is not limited to FcγRI (CD64), including isoforms FcγRIa, FcγRIb, and FcγRIc; FcγRIIa (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2), as well as any undiscovered human FcγRs or FcγR isoforms or allotypes. An FcγR can be from any organism, including but not limited to humans, mice, rats, rabbits, and monkeys. Mouse FcγRs include but are not limited to FcγRI (CD64), FcγRIIb (CD32), FcγRIII (CD16), and FcγRIV (CD16-2), as well as any undiscovered mouse FcγRs or FcγR isoforms or allotypes.

The term “FcRn” or “FcRn receptor” as used herein refers to an Fc receptor (“n” indicates neonatal based on the first identified function) which is known to be involved in transfer of maternal IgGs to a fetus through the human or primate placenta, or yolk sac (rabbits, rats and mice) and to a neonate from the colostrum through the small intestine. It is also known that FcRn is involved in the maintenance of constant serum IgG levels by binding the IgG molecules and recycling them into the serum. The binding of FcRn to IgG molecules is pH-dependent with optimum binding at pH 6.0 and weak binding at pH >7.0. Whereas the binding of IgGs to FcγR receptors can trigger effector function (e.g., ADCC), binding to FcRn in a pH dependent manner can prolong the half-life on IgG antibodies in the serum. Effector function can be undesirable for a molecule with a prolonged half-life in serum or that target a soluble version of a protein or receptor also displayed by cells.

The term “effector function” as used herein refers to a biochemical event that results from the interaction of an Fc domain with an Fc receptor or ligand. Effector functions include but are not limited to ADCC, ADCP, and CDC. By “effector cell” as used herein is meant a cell of the immune system that expresses or one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and τδ T cells, and can be from any organism included but not limited to humans, mice, rats, rabbits, and monkeys.

The terms “antibody-dependent cell-mediated cytotoxicity” or “ADCC” refer to a form of cytotoxicity in which a polypeptide comprising an Fc domain, e.g., an antibody, bound onto Fc receptors (FcRs) present on certain cytotoxic cells (e.g., primarily NK cells, neutrophils, and macrophages) and enables these cytotoxic effector cells to bind specifically to an antigen-bearing “target cell” and subsequently kill the target cell with cytotoxins. (Hogarth et al., Nature review Drug Discovery 2012, 11: 313) It is contemplated that, in addition to antibodies and fragments thereof, other polypeptides comprising Fc domains, e.g., Fc fusion proteins and Fc conjugate proteins, having the capacity to bind specifically to an antigen-bearing target cell will be able to effect cell-mediated cytotoxicity.

For simplicity, the cell-mediated cytotoxicity resulting from the activity of a polypeptide comprising an Fc domain is also referred to herein as ADCC activity. The ability of any particular polypeptide of the present disclosure to mediate lysis of the target cell by ADCC can be assayed. To assess ADCC activity, a polypeptide of interest (e.g., an antibody) is added to target cells in combination with immune effector cells, resulting in cytolysis of the target cell. Cytolysis is generally detected by the release of label (e.g., radioactive substrates, fluorescent dyes or natural intracellular proteins) from the lysed cells. Useful effector cells for such assays include peripheral blood mononuclear cells (PBMC) and Natural Killer (NK) cells.

The term “ADCP” as used herein refers to antibody directed cellular phagocytosis, i.e., provides mechanisms for clearance of virus and virus-infected cells, as well as for stimulation of downstream adaptive immune responses by facilitating antigen presentation, or by stimulating the secretion of inflammatory mediators. The term “CDC” as used herein refers to complement dependent cytotoxicity, i.e., a biochemical event of targeted cell destruction mediated by the complement system.

The terms “half-life” or “in vivo half-life” as used herein refer to the biological half-life of a particular type of polypeptide of the present disclosure in the circulation of a given animal and is represented by a time required for half the quantity administered in the animal to be cleared from the circulation and/or other tissues in the animal.

The term “subject” as used herein refers to any animal (e.g., a mammal), including, but not limited to humans, non-human primates, rodents, and the like, which is to be the recipient of a particular treatment. The terms “subject” and “patient” are used interchangeably herein in reference to a human subject.

The term “pharmaceutical composition” as used herein refers to a preparation which is in such form as to permit the biological activity of the active ingredient to be effective, and which contains no additional components which are unacceptably toxic to a subject to which the composition would be administered. Such composition can be sterile.

An “effective amount” of a polypeptide, e.g., an antibody, as disclosed herein is an amount sufficient to carry out a specifically stated purpose. An “effective amount” can be determined empirically and in a routine manner, in relation to the stated purpose. The term “therapeutically effective amount” as used herein refers to an amount of a polypeptide, e.g., an antibody, or other drug effective to “treat” a disease or disorder in a subject or mammal.

The term “label” when used herein refers to a detectable compound or composition which is conjugated directly or indirectly to a polypeptide, e.g., an antibody, so as to generate a “labeled” polypeptide. The label can be detectable by itself (e.g., radioisotope labels or fluorescent labels) or, in the case of an enzymatic label, can catalyze chemical alteration of a substrate compound or composition which is detectable.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented.

The treatment or prevention of a disorder of pregnancy in a pregnant woman as described herein refers to treatment of prevention of a disorder in a pregnant woman, and is not intended to cover treatment of pregnancy per se.

The term “vector” means a construct, which is capable of delivering, and in some aspects, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

### Variant IgG Fc Domains

In some aspects, variant IgG Fc domains are provided which have mutations that confer reduced maternofetal transfer, preferably by more than 90%, more preferably by more than 95% and more preferably by more than 98% when compared to the same polypeptide comprising a parent Fc domain (which may be a wild type domain). These variant IgG Fc domains can be introduced to therapeutic antibodies to ensure safety of the fetus while treating pregnant women.

Although FcRn plays a major role in IgG transfer, the inventors made the hypothesis that other receptors were likely involved since FcRn is not present on the fetal endothelium—which would be expected for a FcRn-only transport process—and maternofetal IgG selective transfer depending on glycosylation or antigen specificity has been reported—which does not reflect selectivity of FcRn binding (Jennewein, M. F., et al, Fc Glycan-Mediated Regulation of Placental Antibody Transfer. Cell. 2019, 27: 202-215).

The inventors then investigated the disruption of the Fc—FcγR binding. The FcγRIIb which like FcRn can bind monomeric IgG, is the sole Fc receptor localised to the fetal placental endothelium and, in the absence of FcRn, is reported to be responsible for IgG transfer to the fetal circulation (Ishikawa, T., et al, FcγRIIb participates in maternal IgG trafficking of human placental endothelial cells. Int. J. Mol. Med. 2015, 35: 1273-89). The FcγRIIIa is detected in the maternal syncytiotrophoblast membrane and is implicated in the selective transfer of digalactosylated IgG, and possibly IgG-3, which poorly binds FcRn, across the placenta (Jennewein, M. F., et al, Fc Glycan-Mediated Regulation of Placental Antibody Transfer. Cell. 2019, 178: 202-215).

The mutations I253A, H310A or H435A disrupt the binding of FcRn to the Fc region and have been shown individually to inhibit maternofetal transfer by no more than 90%. The triple mutation I253A, H301A and H435A in an erythropoietin-Fc fusion protein inhibited transfer across the neonatal mouse intestine and across the lung by up to 50%. This is the first report of the use of all three mutations in the same Fc construct (Spiekermann, G. M., et al, J Exp Med. 2002, 196:303-10). The triple mutation I253A, H301A and H435A has not been tested for the inhibition of the maternofetal transfer of a modified IgG antibody in the prior art.

FcγR binding sites are located in the CH2 domain of the Fc region and binding can be blocked or greatly reduced by known mutation combinations.

Introduction of the mutations L234A and L235A into the Fc region of IgG are known to greatly reduce binding by the FcγR receptors tested (Hezareh, M., et al, J Virol. 2001, 75:12161-8.).

Other modifications known to greatly reduce FcγR binding include the insertion of arginine after G236 with the additional change of L328 to arginine (Chu, S. Y., et al, Molecular Immunology 2008, 45: 3926-33.) or the changes L234F/L235E/P331S (Oganesyan, V., et al Acta Crystallogr D Biol Crystallogr. 2008, 64: 700-4).

Accordingly, in some aspects a polypeptide is provided which comprises a human variant IgG Fc domain comprising amino acid substitutions numbered according to the Kabat EU index numbering system, relative to a human wild-type Fc domain, wherein:
a) (i) positions 234 and 235 are each substituted with alanine or
   (ii) positions 234 and 235 are each substituted with alanine and position 331 is substituted with serine or
   (iii) position 234 is substituted with phenylalanine, position 235 is substituted with glutamic acid and position 331 is substituted with serine or
   (iv) position 328 is substituted with arginine and arginine is inserted after position 236;
and
b) (i) position 253 is substituted with alanine or
   (ii) position 435 is substituted with alanine or
   (iii) positions 235, 310 and 435 are substituted with alanine or
   (iv) position 310 is substituted with alanine and position 435 is substituted with glutamine

In some aspects a polypeptide is provided which comprises a Human variant IgG-1 Fc domain comprising an amino acid sequence that is at least 80%, preferably at least 90% identical to the amino acid sequence of SEQ ID NO: 5 and is comprising amino acid substitutions numbered according to the Kabat EU index numbering system, wherein:
a) (i) positions 234 and 235 are each substituted with alanine or
   (ii) positions 234 and 235 are each substituted with alanine and position 331 is substituted with serine or
   (iii) position 234 is substituted with phenylalanine, position 235 is substituted with glutamic acid and position 331 is substituted with serine or
   (iv) position 328 is substituted with arginine and arginine is inserted after position 236;
and
b) (i) position 253 is substituted with alanine or
   (ii) position 435 is substituted with alanine or
   (iii) positions 235, 310 and 435 are substituted with alanine or
   (iv) position 310 is substituted with alanine and position 435 is substituted with glutamine

The resulting variant Ig Fc domains polypeptide has reduced binding to at least one Fcγ receptor (FcγR) and to FcRn when compared to the same polypeptide comprising a parent Fc domain (which may be a wild type domain).

The Ig Fc domain can have the same modifications in both polypeptides, modification only in one of the two polypeptides which comprise the Fc domain or different substitutions in each chain. Identical modifications in both polypeptides of the Fc domain is preferred.

In one embodiment, the resulting variant Ig Fc domains polypeptide exhibits an increase of at least 2 fold, at least 5 fold or at least 10 fold in the concentration of antibody needed to give 50% binding, or activation, in a binding or cellular activation assay and/or a decrease in the Km binding constant by a factor of at least 2 fold, at least 5 fold or at least 10 fold by SPR or equivalent method to at least one Fcγ receptor and to FcRn when compared to the same polypeptide comprising a parent Fc domain (which may be a wild type domain).

In one embodiment, at least one FcγR is selected from FcγRI, FcγRIIa, FcγRIIb, FcγRIIIa and FcγRIIIb, preferably FcγRIIb or FcγRIIIa.

As such, the resulting polypeptide preferably shows reduced maternofetal transfer when compared to the same polypeptide comprising the parent Fc domain (which may be a wild type domain), preferably by more than 90%, more preferably by more than 95%, more preferably by more than 98% when compared to the same polypeptide comprising the parent Fc domain (which may be a wild type domain) but also display a shorter half-life which is also a safety advantage since treatment can be interrupted if important safety issues arise and the antibody is quickly eliminated.

In one aspect the polypeptides, antibodies, and compositions of the invention can be dosed at therapeutically effective doses more frequently than polypeptides and antibodies which lack the mutations as disclosed herein, for example can be dosed once a day, once every 2, 3, 4, 5, 6 days or every week.

In one aspect, a polypeptide is provided which comprises a human variant IgG Fc domain, which comprises an alanine (A) at positions 234 and 235 in order to greatly reduce FcγR binding and comprises alanine (A) at positions 253, 310 and 435 in order to greatly reduce FcRn binding. Hereinafter, this variant IgG Fc domain and set of amino acid substitutions will be referred to as “L234A/L235A+I253A/H301A/H435A”.

In another aspect, a polypeptide is provided which comprises a human variant IgG Fc domain, which comprises alanine (A) at positions 234 and 235 and serine (S) at position 331 and comprises alanine (A) at positions 253, 310 and 435. Hereinafter, this variant IgG Fc domain and set of amino acid substitutions will be referred to as “L234A/L235A/P331S+I253A/H301A/H435A”.

In another aspect, a polypeptide is provided which comprises a human variant IgG Fc domain, which comprises phenylalanine (F) at position 234, glutamic acid (E) at 235 and serine (S) at position 331 and comprises alanine (A) at positions 253, 310 and 435. Hereinafter, this variant IgG Fc domain and set of amino acid substitutions will be referred to as “L234F/L235E/P331S+I253A/H301A/H435A”.

In another aspect, a polypeptide is provided which comprises a human variant IgG Fc domain, which comprises an arginine (R) inserted after position 236 and an arginine (R) at position 328 and comprises alanine (A) at positions 253, 310 and 435. Hereinafter, this variant IgG Fc domain and set of amino acid substitutions will be referred to as “\hat{ }236R/L328R+I253A/H301A/H435A”.

In another aspect, a polypeptide is provided which comprises a human variant IgG Fc domain, with any of one the FcγR binding reduction mutation sets L234A/L235A, L234A/L235A/P331S, L234F/L235E/P331S or \hat{ }236R/L328R which comprises alanine (A) at position 253. Hereinafter, this variant IgG Fc domain and set of amino acid substitutions will be referred to as a combination of the FcγR mutation set and “I253A”, for example “L234A/L235A/P331S+I253A”.

In another aspect, a polypeptide is provided which comprises a human variant IgG Fc domain, with any of one the FcγR binding reduction mutation sets L234A/L235A, L234A/L235A/P331S, L234F/L235E/P331S or \hat{ }236R/L328R which comprises alanine (A) at position 435. Hereinafter, this variant IgG Fc domain and set of amino acid substitutions will be referred to as a combination of the FcγR mutation set and “H435A”, for example “L234A/L235A/P331S+H435A”.

In another aspect, a polypeptide is provided which comprises a human variant IgG Fc domain, with any of one the FcγR binding reduction mutation sets L234A/L235A, L234A/L235A/P331S, L234F/L235E/P331S or\hat{ }236R/L328R which comprises alanine (A) at position 310 and glutamine (Q) at position 435. Hereinafter, this variant IgG Fc domain and amino acid substitution will be referred to as a combination of the FcγR mutation set and “H301A/H435Q”, for example “L234A/L235A/P331S+H301A/H435Q”.

Thus a set of polypeptides are provided which comprise a human variant IgG Fc domain with any combination of one of the four amino acid substitution sets L234A/L235A or L234A/L235A/P331S or L234F/L235E/P331S or \hat{ }236R/L328R with any one of the four amino acid substitutions or substitution sets I253A/H301A/H435A or I253A or H435A or H301A/H435Q. In some aspects, the parent polypeptide of the variant IgG Fc domain already contains one or more of the amino acids corresponding to the substitutions discussed above, e.g., the parent Fc polypeptide can contain a phenylalanine (F) at position 234 as is found in IgG4. In such aspects, no modification of the amino acid or amino acids already containing one or more of the disclosed substitutions is required.

In some aspects, the variant IgG Fc domain is human. In some other aspects, the variant IgG Fc domain is non-human. Non-human IgG Fc domains can be, e.g., from rodents (e.g., rats or mice), donkey, sheep, rabbit, goat, guinea pig, camel, horse, or chicken. Preferably, the IgG Fc domain is selected from the group consisting of human immunoglobulin G class 1 (IgG1) Fc domain, human immunoglobulin G class 2 (IgG2) Fc domain, human immunoglobulin G class 3 (IgG3) Fc domain, and human immunoglobulin G class 4 (IgG4) Fc domain, preferably human immunoglobulin G class 1 (IgG1) Fc domain. When the variant IgG Fc domain is a mouse IgG Fc domain, the domain can be, e.g., a subclass IgG1, IgG2a, IgG2b, or IgG3 domain.

In some aspects, a polypeptide is provided which comprises a variant IgG Fc domain comprising an amino acid sequence selected from the group consisting of SEQ ID NO:5 to SEQ ID NO:9. SEQ ID NO:5 is a naturally occurring ‘wild type’ sequence corresponding to the Human IgG1 allotype G1m3. SEQ ID NO:6 to SEQ ID NO:9 comprise variants of SEQ ID NO:5 which include mutations or mutation sets which greatly reduce FcγRs and/or FcRn binding as follows: SEQ ID NO:6: L234A/L235A/P331S and I253A/H301A/H435A, SEQ ID NO:7: L234A/L235A/P331S and H301A/H435Q, SEQ ID NO: 8: L234F/L235E/P331S and I253A/H301A/H435A and SEQ ID NO:9: L234F/L235E/P331S and H301A/H435Q.

In some aspects, a polypeptide is provided which comprises a variant IgG Fc domain comprising an amino acid sequence that is at least 80%, preferably at least 90%, more preferably at least 95% identical to the amino acid sequence selected from the group consisting of SEQ ID NO:5 to SEQ ID NO:9, suitably when considered over the length of SEQ ID NO:5 to SEQ ID NO:9, respectively

In some other aspects, a polypeptide is provided which comprises a variant IgG Fc domain consisting of an amino acid sequence selected from the group consisting of SEQ ID NO:5 to SEQ ID NO:9. Based on the teaching provided herein, it will be understood by one of skill in the art that the variant IgG Fc domains provided in SEQ ID NO:5 to SEQ ID NO:9 represent one particular allelic variation. Accordingly, in some aspects, a polypeptide is provided which comprises a different allelic variation of a variant IgG Fc domain as provided in SEQ ID NO:5 to SEQ ID NO:9. Sites of known allelic variation are available in the literature and various databases, for example: (Jefferies, R., et al, Human immunoglobulin allotypes: Possible implications for immunogenicity, mAbs 2009, 1: 332-338 and IMGT database: www.imgt.org).

### Binding to Fc Receptors

A polypeptide comprising a variant IgG Fc domain provided herein (e.g., an antibody or fragments thereof comprising a variant IgG Fc domain) results in reduced or ablated binding for at least one FcγR receptor (e.g., FcγR IIb, FcγR IIIa) and the FcRn receptor.

One skilled in the art will understand that a polypeptide comprising a variant IgG Fc domain can have altered (relative to an unmodified molecule) FcγR and FcRn binding properties. Examples of binding properties include but are not limited to, binding specificity, dissociation and association rates (koff and kon, respectively), equilibrium dissociation constant (KD, defined as the ratio of koff divided by kon), binding affinity and/or avidity.

The affinities and binding properties of a polypeptide comprising a variant IgG Fc domain for a receptor or ligand, can be determined by a variety of in vitro assay methods (biochemical or immunological based assays) known in the art for determining Fc-FcγR and Fc-FcRn interactions, i.e., specific binding of an Fc region to an FcγR. Such methods include equilibrium methods (e.g., enzyme-linked immunoabsorbent assay (ELISA) or radioimmunoassay (RIA)), or kinetics (e.g., surface plasma resonance, such as BIACORE® analysis), and other methods such as indirect binding assays, competitive inhibition assays, fluorescence resonance energy transfer (FRET), gel electrophoresis and chromatography (e.g., gel filtration).

These and other methods can utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999).

In one aspect, a polypeptide comprising a variant IgG Fc domain is provided which exhibits reduced binding affinity for at least one Fcγ receptors including, but not limited to FcγRI (including isoforms FcγRIa, FcγRIb, and FcγRIc); FcγRII (including isoforms FcγRIIa, FcγRIIb, and FcγRIIc); and FcγRIII (including isoforms FcγRIIIa and FcγRIIIb) and for the FcRn receptor as compared to a parent polypeptide comprising a wild type or modified wt Fc domain. In another aspect, the binding of a polypeptide comprising a variant IgG Fc domain to one or more Fcγ receptors and FcRn as noted above is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than a parent polypeptide comprising a wild type or modified wt Fc domain or is reduced to an undetectable level.

In another aspect, the binding of a polypeptide comprising a variant IgG Fc domain to one or more Fcγ receptors and FcRn as noted above is fully ablated.

In one aspect, a polypeptide comprising a variant IgG Fc domain is provided which exhibits a decreased affinity to FcγRI relative to a parent polypeptide comprising a wild type or modified wt Fc domain. In another aspect, a polypeptide comprising a variant IgG Fc domain is provided which exhibits an affinity for FcγRI receptor that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than a parent polypeptide comprising a wild type or modified wt Fc domain or is reduced to an undetectable level.

In another aspect, a polypeptide comprising a variant IgG Fc domain is provided which exhibits an affinity for FcγRI receptor that is at least 90%, at least 80%, at least 70%, at least 60%, at least 50% less than a parent polypeptide comprising a wild type or modified wt Fc domain. In some aspects, the FcγRI is isoform FcγRIa. In other aspects, the FcγRI is isoform FcγRIb. In yet another aspect, the FcγRI is isoform FcγRIc.

In one aspect, a polypeptide comprising a variant IgG Fc domain is provided which exhibits a decreased affinity to FcγRII relative to a parent polypeptide comprising a wild type or modified wt Fc domain. In another aspect, a polypeptide comprising a variant IgG Fc domain is provided which exhibits an affinity for FcγRII receptor that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than a parent polypeptide comprising a wild type or modified wt Fc domain.

In another aspect, a polypeptide comprising a variant IgG Fc domain is provided which exhibits an affinity for FcγRII receptor that is at least 90%, at least 80%, at least 70%, at least 60%, at least 50% less than a parent polypeptide comprising a wild type or modified wt Fc domain. In some aspects, the FcγRII is isoform FcγRIIa. In another aspect, the FcγRIIa isoform is allotype H131. In yet another aspect, the FcγRIIa isoform is allotype R131. In other aspects, the FcγRII is isoform FcγRIIb. In some aspects, the FcγRIIb isoform is FcγRIIb-1. In other aspects, the FcγRIIb isoform is FcγRIIb-2. In yet another aspect, the FcγRII is isoform FcγRIIc.

In one aspect, a polypeptide comprising a variant IgG Fc domain is provided which exhibits a decreased affinity to FcγRIII relative to a parent polypeptide comprising a wild type or modified wt Fc domain. In another aspect, a polypeptide comprising a variant IgG Fc domain is provided which exhibits an affinity for FcγRIII receptor that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than a parent polypeptide comprising a wild type or modified wt Fc domain.

In another aspect, a polypeptide comprising a variant IgG Fc domain is provided which exhibits an affinity for FcγRIII receptor that is at least 90%, at least 80%, at least 70%, at least 60%, at least 50%, less than a parent polypeptide comprising a wild type or modified wt Fc domain. In some aspects, the FcγRIII is isoform FcγRIIIa. In other aspects, the FcγRIIIa is allotype 158V (F158V allelic variant). In other aspects, the FcγRIIIa is allotype 158F. In other aspects, the FcγRIII is isoform FcγRIIIb. In another aspect, the FcγRIIIb is allotype NA1. In other aspects, the FcγRIIIb is allotype NA2.

In one aspect, a polypeptide comprising a variant IgG Fc domain is provided which exhibits a decreased affinity to FcRn relative to a parent polypeptide comprising a wild type or modified wt Fc domain. In another aspect, a polypeptide comprising a variant IgG Fc domain is provided which exhibits an affinity for FcRn receptors that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold less than a parent polypeptide comprising a wild type or modified wt Fc domain. In another aspect, a polypeptide comprising a variant IgG Fc domain is provided which exhibits an affinity for FcRn receptors that is at least 90%, at least 80%, at least 70%, at least 60%, at least 50%, less than a parent polypeptide comprising a wild type or modified wt Fc domain.

In one aspect, a polypeptide comprising a variant IgG Fc domain is provided which exhibits an affinity for FcγR receptors, as measured by the dissociation constant (koff divided by kon, KD) that is between about 100 nM to about 100 μM, or about 100 nM to about 10 μM, or about 100 nM to about 1 μM, or about 1 nM to about 100 μM, or about 10 nM to about 100 μM, or about 1 μM to about 100 μM, or about 10 μM to about 100 μM. In certain aspects, a polypeptide comprising a variant IgG Fc domain is provided which exhibits an affinity for FcγR receptors that is greater than 1 μM, greater than 5 μM, greater than 10 μM, greater than 25 μM, greater than 50 μM, or greater than 100 μM. In another aspect, a polypeptide comprising a variant IgG Fc domain is provided which exhibits an affinity for FcγR receptors that is less than 100 μM, less than 50 μM, less than 10 μM, less than 5 μM, less than 2.5 μM, less than 1 μM, or less than 100 nM, or less than 10 nM.

In one aspect, a polypeptide comprising a variant IgG Fc domain is provided which exhibits an affinity for FcRn receptors that is between about 100 nM to about 100 μM, or about 100 nM to about 10 μM, or about 100 nM to about 1 μM, or about 1 nM to about 100 μM, or about 10 nM to about 100 μM, or about 1 μM to about 100 μM, or about 10 μM to about 100 μM. In certain aspects, a polypeptide comprising a variant IgG Fc domain is provided which exhibits an affinity for FcRn receptors that is greater than 1 μM, greater than 5 μM, greater than 10 μM, greater than 25 μM, greater than 50 μM, or greater than 100 μM. In another aspect, a polypeptide comprising a variant IgG Fc domain is provided which exhibits an affinity for FcRn receptors that is less than 100 μM, less than 50 μM, less than 10 μM, less than 5 μM, less than 2.5 μM, less than 1 μM, or less than 100 nM, or less than 10 nM.

In specific aspects, a polypeptide comprising L234A/L235A or L234A/L235A/P331S or L234F/L235E/P331S or \hat{ }236R/L328R substitutions in the IgG Fc domain which exhibits a decreased affinity to FcγR which further comprises I253A/H301A/H435A or I253A or H435A or H301A/H435Q substitutions in the IgG Fc domain is provided which exhibits a decreased affinity to FcRn as compared to a parent polypeptide comprising a wild type or modified wt IgG Fc domain.

In specific aspects, a polypeptide comprising L234A/L235A or L234A/L235A/P331S or L234F/L235E/P331S or \hat{ }236R/L328R substitutions in the IgG Fc domain which exhibits a fully ablated binding to FcγR which further comprises I253A/H301A/H435A or I253A or H435A or H301A/H435Q substitutions in the IgG Fc domain is provided which exhibits a decreased affinity to FcRn as compared to a parent polypeptide comprising a wild type or modified wt IgG Fc domain.

In specific aspects, a polypeptide comprising L234A/L235A or L234A/L235A/P331S or L234F/L235E/P331S or \hat{ }236R/L328R substitutions in the IgG Fc domain which exhibits a decreased affinity to FcγR which further comprises I253A/H301A/H435A or I253A or H435A or H301A/H435Q substitutions in the IgG Fc domain is provided which exhibits a fully ablated binding to FcRn as compared to a parent polypeptide comprising a wild type or modified wt IgG Fc domain.

In specific aspects, a polypeptide comprising L234A/L235A or L234A/L235A/P331S or L234F/L235E/P331S or \hat{ }236R/L328R substitutions in the IgG Fc domain which exhibits a fully ablated binding to FcγR which further comprises I253A/H301A/H435A or I253A or H435A or H301A/H435Q substitutions in the IgG Fc domain is provided which exhibits a fully ablated binding to FcRn as compared to a parent polypeptide comprising a wild type or modified wt IgG Fc domain.

In one aspect the invention relates to a molecule, such as an antibody or fragment thereof, which comprises a first polypeptide and a second polypeptide each comprising, in N-terminal to C-terminal direction at least a portion of an immunoglobulin hinge region, which comprises one or more cysteine residues, an immunoglobulin CH2-domain and an immunoglobulin CH3-domain, wherein the first and the second polypeptides each comprise a mutation(s) from group A below and a mutation(s) from group B below,
A
(i) positions 234 and 235 are each substituted with alanine or
(ii) positions 234 and 235 are each substituted with alanine and position 331 is substituted with serine or
(iii) position 234 is substituted with phenylalanine, position 235 is substituted with glutamic acid and position 331 is substituted with serine or
(iv) position 328 is substituted with arginine and arginine is inserted after position 236;
and
B
(i) position 253 is substituted with alanine or
(ii) position 435 is substituted with alanine or
(iii) positions 235, 310 and 435 are substituted with alanine or
(iv) position 310 is substituted with alanine and position 435 is substituted with glutamine

And wherein the first and second polypeptide may have the same or different mutations(s) (preferably the same), and wherein the mutations are defined with respect to the wild type or modified wt sequence as defined herein.

All features of the present disclosure apply to the above aspect

### Methods

In some aspects, a method to inhibit the maternofetal transfer ability in a parent polypeptide comprising an Fc domain comprising the steps of:
a) (i) positions 234 and 235 are each substituted with alanine or
   (ii) positions 234 and 235 are each substituted with alanine and position 331 is substituted with serine or
   (iii) position 234 is substituted with phenylalanine, position 235 is substituted with glutamic acid and position 331 is substituted with serine or
   (iv) position 328 is substituted with arginine and arginine is inserted after position 236;
and
b) (i) position 253 is substituted with alanine or
   (ii) position 435 is substituted with alanine or
   (iii) positions 235, 310 and 435 are substituted with alanine or
   (iv) position 310 is substituted with alanine and position 435 is substituted with glutamine

### Antibodies and Fragments Thereof

In some aspects, a polypeptide comprising a variant IgG Fc domain comprises an antigen binding domain. In some specific aspects, the antigen-binding domain can be an antibody, e.g., a monoclonal antibody, or an antigen-binding fragment thereof. The antigen-binding domain can be a full-length antibody, e.g., a human antibody, a humanized antibody, or a chimeric antibody, or a fragment thereof.

The term “antibody variant” refers to a polypeptide containing a variant IgG Fc domain provided herein, wherein the polypeptide is an antibody. Antibody variants include monoclonal antibodies, multispecific antibodies, human antibodies, humanized antibodies, camelized antibodies, chimeric antibodies, anti-idiotypic (anti-Id) antibodies, and Fc domain-containing fragments of any of the above. In some aspects, antibody variants include immunoglobulin molecules and immunologically active fragments of immunoglobulin molecules, i.e., molecules that contain an antigen binding site, wherein these fragments can be fused or conjugated to another immunoglobulin domain comprising a variant IgG Fc domain provided herein. In one aspect, the antibody variants are of the human IgG1, IgG2, IgG3 or IgG4 isotype.

Antibody variants and fragments thereof comprising a variant IgG Fc domain provided herein can be from any animal origin including birds and mammals (e.g., human, a rodent such as mouse or rat, donkey, sheep, rabbit, goat, guinea pig, camel, horse, or chicken). In a specific aspect, an antibody variant is provided which is a human or a humanized monoclonal antibody. As used herein, “human” antibodies include antibodies having the amino acid sequence of a human immunoglobulin and also include antibodies isolated from human immunoglobulin libraries, synthetic immunoglobulin libraries in microorganisms or from mice or other birds or mammals that express antibodies from human genes.

An antibody variant can be monospecific, bispecific, trispecific or have greater specificity (multispecific antibodies). Multispecific antibody variants can specifically bind to different epitopes of desired target molecule or can specifically bind to both the target molecule as well as a heterologous epitope, such as a heterologous polypeptide or solid support material.

### Specific Therapeutic Targets

Virtually any molecule can be targeted by a binding-molecule, e.g., an antibody, fusion protein, or conjugate comprising a variant IgG Fc domain according to the present invention. In additional, virtually any molecule can be incorporated into a fusion protein or a conjugate comprising a variant IgG Fc domain provided herein. However, as explained previously the variant IgG Fc domain of the present invention can be particularly useful for treating pregnant women.

Pregnancy comes with risks of severe complications and mobidity, even mortality. The Centers for Disease Control and Prevention (CDC) lists the following severe morbidity conditions: Acute myocardial infarction, Aneurysm, Acute renal failure, Adult respiratory distress syndrome, Amniotic fluid embolism, Cardiac arrest/ventricular fibrillation, Conversion of cardiac rhythm, Disseminated intravascular coagulation, Eclampsia, Heart failure/arrest during surgery or procedure, Puerperal cerebrovascular disorders, Pulmonary edema/Acute heart failure, Severe anesthesia complications, Sepsis and Shock (www.cdc.gov/reproductivehealth/maternalinfanthealth/severematemalmorbidity.html). Even where treatments are available for these conditions in the population in general they are most likely not suitable, or proven safe, for the treatment of pregnant women due to the risk of maternofetal transfer and the potential adverse effects on the fetus. Many of the morbidities of pregnancy could be treated safely and effectively using antibodies, fusion proteins or conjugates comprising a variant IgG Fc domain according to the present invention.

Hypertension is a common condition in pregnancy and the underlying condition responsible for many of the morbidities of pregnancy. According to the Center for Disease Control about 10% of mothers hospitalized in 2004 in the US suffered hypertension. Hypertension during pregnancy can result in life-long circulatory damage, seizures and stroke in the mother and severe problems for the baby resulting from pre-term birth and low-birth weight. Hypertension, and pre-eclampsia/eclampsia in particular, are major causes of morbidity and mortality for mother and babies across the world and new treatments are clearly needed (Ives, C. W., et al, J. Am Coll Cardiol. 2020, 76: 1690-1702).

Hypertension, and pre-eclampsia/eclampsia, as well as other disorders of pregnancy seen in pregnant women, are well known and described in the common general knowledge textbooks such as, for example:
Sankaran S. Creasy and Resnik's Maternal-Fetal Medicine: Principles and Practice Sixth edition. Obstet Med. 2012 June; 5(2):88-9. doi: 10.1258/om.2011.11E005. Epub 2012 Jun. 19. PMCID: PMC4989620.
Magee, L. A., von Dadelszen, P., Stones, W., & Mathai, M. (2016). The FIGO Textbook of Pregnancy Hypertension: an evidence-based guide to monitoring, prevention and management.
Gestational Hypertension and Preeclampsia: ACOG Practice Bulletin, Number 222. Obstetrics & Gynecology 135(6):p e237-e260, June 2020. 1 DOI: 10.1097/AOG.0000000000003891
Saito, Shigeru 2018. Preeclampsia Basic, Genomic, and Clinical: Basic, Genomic, and Clinical DOI—10.1007/978-981-10-5891-2

The invention thus relates to any therapeutic agent described herein, such as polypeptides and antibodies, with an Fc modification as described and claimed herein, for the treatment in a pregnant woman of any one of: Bacterial and Parasitic Infections; Maternal and Fetal Viral Infections; Sexually Transmitted Diseases; Maternal-Fetal Infections; Cardiac Diseases; Coagulation Disorders; Thromboembolic Disease; Anemia; Malignancy; Renal Disorders; Respiratory Diseases; Diabetes; Thyroid Disease and other Endocrine Disorders; Gastrointestinal Disease; Diseases of the Liver, Biliary System, Pancreas; and Rheumatic Diseases.

These disorders found in pregnant women are described in Creasy and Resnik (above), which list of disorders and treatments is specifically incorporated by reference herein.

Hypertension complicates 5 to 10% of pregnancies and hypertensive disorders of pregnancy can be categorized as chronic, gestational or pre-eclamptic. Pre-eclampsia is especially serious being one of the top five causes of maternal and perinatal mortality. In 2005 it was estimated that world-wide pre-eclampsia claimed the lives of more than 70,000 women per year and more than 500,000 of their fetuses and newborns (Sabai, B., et al, Lancet 2005, 365: 785-99).

Many compounds are available for the treatment of hypertension in the general population: in 2011 the FDA listed 69 approved drugs from 15 different drug classes (www.fda.gov/regulatory-information/search-fda-guidance-documents/hypertension-indication-drug-labeling-cardiovascular-outcome-claims). However, none of these are specifically tested and approved for use during pregnancy and most, if not all, are small molecules or small peptide drugs which have a high possibility of crossing the placenta with unknown effects on the fetus.

Conventional medicines to control hypertension are sometimes used for the treatment of pregnant women but all of these have maternal or fetal risks, limited effectiveness, especially in the treatment of pre-eclampsia, and require a challenging risk-benefit evaluation with a severely limited set of data. Thus, there is a clear unmet need for therapies which treat hypertension and pre-eclampsia without exposing the fetus to harm or the risk of harm. This problem is solved by providing antibodies, fusion proteins or conjugates comprising a human variant IgG Fc domain according to the invention which can be developed to target and affect soluble proteins, receptors or small molecules that reduce, modulate or abolish or otherwise beneficially interfere with mechanisms of hypertension and pre-eclampsia.

Currently available drugs to treat hypertension include Diuretics, Beta-blockers, ACE inhibitors, Angiotensin II receptor blockers, Calcium channel blockers, Alpha blockers Alpha-2 Receptor Agonists, Combined alpha and beta-blockers, Central nervous system agonists, Peripheral adrenergic inhibitors and Vasodilators. New drug classes including inhibitors of vasopeptidases, aldosterone synthase and soluble epoxide hydrolase, agonists of natriuretic peptide A and vasoactive intestinal peptide receptor 2, and a novel mineralocorticoid receptor antagonist are in phase II/III clinical development, while inhibitors of aminopeptidase A, dopamine β-hydroxylase, and the intestinal Na+/H+ exchanger 3, agonists of components of the angiotensin converting enzyme 2/angiotensin(1-7)/Mas receptor axis are in earlier stage development. Many of the underlying mechanisms of action of these classes of drugs can be amenable to the development of antibodies, fusion proteins or conjugates comprising a variant IgG Fc domain according to the present invention and thus safe for the treatment of pregnant women.

Treatment for Pre-Eclampsia Targeting sVEGFR-1

Pre-eclampsia is a disorder of pregnancy associated with new-onset hypertension, which occurs most often after 20 weeks of gestation and frequently near term. Several mechanisms of disease have been proposed in preeclampsia including the following: chronic uteroplacental ischemia, immune maladaptation, very low-density lipoprotein toxicity, increased trophoblast apoptosis or necrosis, and an exaggerated maternal inflammatory response to deported trophoblasts. Currently it is believed that imbalances of angiogenic factors are important in the pathogenesis of preeclampsia and a combination of some of the other purported mechanisms may be responsible for triggering the clinical spectrum of preeclampsia. For example, there is clinical and experimental evidence suggesting that uteroplacental ischemia leads to increased circulating concentrations of antiangiogenic factors, including sVEGFR-1 which promote angiogenic imbalances.

The development of the placenta requires a complex interplay of new blood vessels to manage the transfer functions between mother and fetus which must coordinate with the increasing requirements as the fetus grows. Pre-eclampsia is considered to be a consequence of an imbalance in this process where the fetus is not sufficiently served with nutrients and oxygen and the balance of pro- and anti-angiogenic factors is upset. The sequalae of this imbalance can trigger general disruption to the maternal endothelium and invoke inappropriate immune responses. Thus there are many potential targets for antibodies which can treat the mother without impacting the fetus. The anti-angiogenic factor soluble vascular endothelial growth factor receptor-1 (sVEGFR-1) is believed to be of prime importance in the pathogenesis of pre-eclampsia, and thus prime a target for treatment using antibodies comprising a variant IgG Fc domain according to the present invention.

Experimental and epidemiological studies support a pathological role for sVEGFR-1 in the imbalance of circulating angiogenic and anti-angiogenic factors in the aetiology of the maternal syndrome of pre-eclampsia. sVEGFR-1 is a soluble circulating protein which functions to regulate the activities of VEGF and placental derived growth factor (PGF) by sequestration and prevention of binding to functional membrane bound cell surface receptors (Rana, S., et al, Am. J. Obstet. Gynecol. 2022, 226: S1019-S1034). Increased levels of sVEGFR-1 and decreasing levels of PGF are correlated with the onset of pre-eclampsia. Removal of sVEGFR-1 by apheresis is reported to prolong pregnancy in women with pre-eclampsia and shows potential therapeutic modality. Ideally an agent that reduces sVEGFR-1 levels whilst also releasing VEGF and PGF, and thus partially restoring normal levels, is desirable. Normal IgG antibodies which bind sVEGFR-1 have been proposed for the treatment of hypertensive conditions in pregnant women. However, these can cross the placenta and potentially interfere with fetal development by changing the availability of VEGF and PGF. Indeed, Avastin, an antibody which binds and blocks the effects of VEGF, carries a warning against the treatment of pregnant women due to safety concerns over fetal exposure. In the same way an antibody with an unmodified Fc region targeted against sVEGFR-1 would not be suitable for use in pregnant women due to safety concerns for the fetus. On the other hand, a modified placenta-impermeable antibody to sVEGFR-1, ideally one that blocks ligand binding and thus releases bound VEGF or PGF, is well suited to treating conditions of pregnancy especially eclampsia and pre-eclampsia. The therapeutic effect is to bind sVEGFR-1 thereby displacing bound VEGF and PGF which is followed by endosomal degradation of the antibody-sVEGFR-1 complex. In addition, sVEGFR-1 plasma levels are measurable by a number of readily available clinical assays and the antibody is titrated depending on the individual patient sVEGFR-1 levels in a responsive fashion due to the faster clearance which is an additional advantage of inhibiting FcRn binding to the Fc domain.

Excess levels of the anti-angiogenic factor sVEGFR-1, which is produced by the placenta and released into the maternal circulation, induce maternal endothelial dysfunction leading to preeclamptic symptoms. sVEGFR-1 is a soluble splice variant of the membrane-bound receptor VEGFR-1 that binds to the proangiogenic proteins VEGF and PGF; therefore, sVEGFR-1 acts as a ligand trap and antagonizes ligand-mediated angiogenic signaling via the cell surface receptors. In rodents, sVEGFR-1 overexpression produces symptoms of pre-eclampsia, and in humans higher maternal levels of sVEGFR-1 are associated with more severe forms of the disease. A high plasma level of sVEGFR-1 and/or a high sVEGFR-1 to PGF ratio indicate that inhibition of angiogenic processes are strong predictors of pre-eclampsia severity and adverse clinical outcomes. Drugs that inhibit angiogenic signaling such as the VEGF sequestering antibody bevacizumab (Avastin; Genentech), the VEGF-trap (aflibercept; Regeneron) and small molecule inhibitors of VEGF receptors are associated with major adverse effects of pre-eclampsia-like symptoms including hypertension, proteinuria and renal glomerular changes in non-pregnant women. Together, these findings indicate that high levels of circulating sVEGFR-1 and low levels of circulating proangiogenic factors (VEGF and PGF) produce an anti-angiogenic state that contributes to the clinical manifestations of pre-eclampsia.

As such, in one aspect, the polypeptide comprising a Human variant IgG Fc domain according to the invention is an anti-VEGFR-1 antibody which inhibits binding of VEGF and/or PGF to sVEGFR-1. In this aspect the anti-sVEGFR-1 antibody binds to the VEGF and/or PGF binding site of sVEGFR-1 with high affinity and thus prevents the sVEGFR-1 from binding VEGF and/or PGF and furthermore releases any already-bound ligand. The antibody thus prevents the binding of VEGF and/or PGF to the sVEGFR-1 receptor and also displaces any bound VEGF and/or PGF thereby abrogating the negative effects of the circulating levels of sVEGFR-1.

IMC-18F (disclosed in U.S. Pat. No. 7,972,596B2, the disclosure of IMC-18F is specifically incorporated by reference) is an IgG1 antibody with high binding affinity to sVEGFR-1, capable of displacing bound VEGF and PDG, which can be modified according to the invention. The heavy chain and light chain sequences are described as SEQ ID NO:11 and SEQ ID NO:12 respectively.

In another aspect, said anti-VEGFR-1 antibody comprises an amino acid sequence that is at least 80%, preferably at least 90%, more preferably at least 95% identical to the amino acid sequence of a heavy chain which comprises a variable heavy chain sequence SEQ ID NO:10 joined to a human variant IgG Fc domain sequence according to the invention, for instance SEQ ID NO: 8, in combination giving the complete heavy chain SEQ ID NO: 13, and a light chain of SEQ ID NO:12 to constitute an IgG-1 which binds to sVEGFR-1 and exhibits maternofetal transfer at very low or undetectable levels.

In another aspect, said anti-VEGFR-1 antibody comprises variable heavy chain sequence SEQ ID NO:10 joined to a human variant IgG Fc domain sequence selected from SEQ ID NO; 6, 7, or, 9 and a light chain of SEQ ID NO:12.

In another aspect, said anti-VEGFR-1 antibody comprises variable heavy chain sequence SEQ ID NO:10 joined to a human variant IgG Fc region comprising a modified IgG-1 sequence (N297A) SEQ ID NO: 14, or comprising an IgG-4 sequence SEQ ID NO:15, or comprising an IgG-4 sequence where serine 228 is substituted with proline SEQ ID NO:16, or comprising an IgG-4 sequence where serine 228 is substituted with proline and leucine 253 is substituted with glutamate SEQ ID NO: 17, or comprising an IgG-2 sequence SEQ ID NO: 18, or comprising a hybrid IgG-2/IgG-4 sequence SEQ ID NO: 19 and a light chain of SEQ ID NO: 13.

In another aspect, the polypeptide comprising a human variant IgG Fc domain according to the invention is an anti-VEGFR-1 antibody comprising an variant Fc domain from the heavy chain SEQ ID NO:5 with any one of the four mutation sets L234A/L235A, L234A/L235A/P331S, L234F/L235E/P331S or \hat{ }236R/L328R designed to reduce FcγR binding in combination with any one of the four mutations or mutation sets I253A/H301A/H435A or 1253A or H435A or H301A/H435Q designed to reduce FcRn binding combined with the heavy chain variable region SEQ ID NO:10 and a light chain of SEQ ID NO:12.

For completeness, a polypeptide comprising a Human variant IgG Fc domain according to the invention can comprise any of the 4 mutation sets L234A/L235A, L234A/L235A/P331S, L234F/L235E/P331S designed to reduce FcγR binding, in combination with any of the 4 mutation sets one of the four mutations or mutation sets I253A/H301A/H435A or 1253A or H435A or H301A/H435Q designed to reduce FcRn binding. The invention therefore relates to a polypeptide comprising any of these 16 individualised mutation sets—optionally in the form of a heavy chain or an antibody.

In another aspect, the polypeptide comprising a Human variant IgG Fc domain according to the invention is an anti-VEGFR-1 antibody comprising an variant Fc domain from any of the heavy chain sequences SEQ ID NO:14, SEQ ID NO:15, SEQ ID NO:16 or SEQ ID NO:17 with any one of the four mutation sets L234A/L235A, L234A/L235A/P331S, L234F/L235E/P331S or \hat{ }236R/L328R designed to reduce FcγR binding (or equivalent changes based on variations in the IgG 4 sequences) in combination with any one of the four mutations or mutation sets I253A/H301A/H435A or I253A or H435A or H301A/H435Q combined designed to reduce FcRn binding (or equivalent changes based on variations in the IgG 4 sequences) with the heavy chain variable region SEQ ID NO:10 and a light chain of SEQ ID NO:12. The equivalent sequence positions and changes for the substitutions between IgG1 and IgG4 sequences are easily determined by one skilled in the art.

In another aspect, the variable heavy chain of the antibody SEQ ID NO: 10 is optimized for lowered immunogenicity potential, stability and manufacturability by one or more of the substitutions alanine-2 substituted with valine, valine-4 substituted with leucine, serine-14 substituted with proline, tryptophan-52 substituted with either serine, tyrosine or phenylalanine, aspartate-53 substituted by alanine, threonine or glutamate or glycine-54 changed to alanine or serine and the selected sequence combined with any of the Human Fc variant regions of the invention, and to light chain SEQ ID NO: 12 to comprise an optimized antibody with both improved therapeutic and manufacturing properties and maternofetal transfer at very low or undetectable levels.

### Other Drug Treatments

The present invention is in particular also applicable to any antibodies or IgG Fc containing molecules known to bind VEGFR1 and which have a therapeutic effect, for example any anti-VEGFR-1 antibody or fragment thereof which inhibits binding of VEGF and/or PGF to sVEGFR-1, or any antibody or fragment thereof as disclosed and claimed in WO2017175054 (referring to eg an antibody or a synthetic or recombinant fragment thereof able to recognize and bind to an epitope comprised in the sequence from aa. 149 to aa. 161 of VEGFR-1 as defined in WO2017175054, such as D16F7—incorporated by reference), and also to the use of such antibodies for the prevention or treatment of disease, such as a disorder of pregnancy, in a pregnant woman.

The invention is also applicable to antibodies or IgG Fc containing polypeptides known to have utility or potential utility in treating disorders of pregnant women, such as Eculizumab (Soliris) for pre-eclampsia.

In another aspect, the approaches and uses described herein also applies to the treatment of a pregnant companion animal, such as dog or cat or horse, or a pregnant livestock animal such as a cow, sheep or goat. Therefore, it will be appreciated that such animals may be treated, and the invention is not limited to pregnant human women.

### Methods of Producing Antibodies Comprising Variant IgG Fc Domains

Antibody variants or fragments thereof can be produced by any method known in the art for the synthesis of antibodies, in particular, by chemical synthesis or by recombinant expression techniques.

Monoclonal antibody variants can be prepared using a wide variety of techniques known in the art including the use of hybridoma, recombinant, and phage display technologies, or a combination thereof. For example, monoclonal antibody variants can be produced using hybridoma techniques including those known in the art. Methods for producing and screening for specific antibodies using hybridoma technology are routine and known in the art.

Antibody variants can be generated by numerous methods well known to one skilled in the art. Non-limiting examples include, isolating antibody coding regions (e.g., from hybridomas) and introducing one or more Fc domain amino acid substitutions into the isolated antibody coding region. Alternatively, the variable regions can be subcloned into a vector encoding a variant IgG Fc domain provided herein.

Antibody variant fragments which recognize specific epitopes can be generated by any technique known to those of skill in the art. For some uses, including in vivo use of antibody variants in humans and in vitro detection assays, it can be advantageous to use human or chimeric antibody variants. Completely human antibodies are particularly desirable for therapeutic treatment of human subjects. Human antibodies or fragments thereof comprising a variant IgG Fc domain provided herein can be made by a variety of methods known in the art.

A chimeric antibody variant or fragment thereof comprising a variant IgG Fc domain provided herein can also be made by a variety of methods known in the art. In certain instances, a humanized antibody variant or fragment thereof can comprise a variant IgG Fc domain provided herein. Humanized antibody variants can be produced using variety of techniques known in the art, including but not limited to, CDR-grafting, veneering or resurfacing, etc.

Human antibody variants can also be produced using transgenic mice which are incapable of expressing functional endogenous immunoglobulins, but which can express human immunoglobulin genes. For example, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen or immunogenic fragments thereof.

Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful antibodies.

### Polynucleotides

A polynucleotide is provided which encodes a polypeptide comprising a variant IgG Fc domain. Also provided is a polynucleotide that hybridizes under high stringency, intermediate, or lower stringency hybridization conditions to a polynucleotide that encodes a polypeptide comprising a variant IgG Fc domain.

In some aspects, a polynucleotide sequence encoding a polypeptide comprising a variant IgG Fc domain can be produced from a parent polynucleotide sequence obtained from a suitable source. Once the polynucleotide sequence has been obtained, the polynucleotide sequence can be manipulated using methods known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate a polypeptide comprising a variant IgG Fc domain having a different amino acid sequence, for example to create amino acid substitutions, deletions, and/or insertions.

In other aspects, a polynucleotide sequence encoding a polypeptide comprising a variant IgG Fc domain can be assembled from chemically synthesized oligonucleotides (e.g., as described in Kutmejer et al. BioTechniques 1994, 17: 242), which, briefly, involves the synthesis of overlapping oligonucleotides containing portions of the encoding sequence, annealing and ligating of those oligonucleotides, and then amplification of the ligated oligonucleotides by PCR.

### Conjugates and Derivatives

In some aspects, a variant IgG Fc domain provided herein can be conjugated or fused to one or more moieties, including but not limited to, peptides, polypeptides, proteins, fusion proteins, nucleic acid molecules, small molecules, mimetic agents, synthetic drugs, inorganic molecules, and organic molecules.

In some aspects, a polypeptide comprising a variant IgG Fc domain includes derivatives that are modified, e.g., by covalent attachment of any type of molecule to the polypeptide or chemical or enzymatic modification. For example, derivatives include polypeptides that have been modified, e.g., by glycosylation, acetylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formylation, etc. Additionally, the derivative can contain one or more non-classical amino acids.

Conjugates are provided which comprise a polypeptide comprising a variant IgG Fc domain chemically conjugated (including both covalent and non-covalent conjugations) to a heterologous protein or polypeptide (or fragment thereof, to a polypeptide of at least 10, at least 20, at least 30, at least 40, at least 50, at least 60, at least 70, at least 80, at least 90 or at least 100 amino acids). The conjugation does not necessarily need to be direct, but can occur through a linker. Such linker molecules are commonly known in the art and described in Denardo et al. Clin Cancer Res 1998, 4:2483; Peterson et al. Bioconjug. Chem. 1999, 10: 553; Zimmerman et al. Nucl. Med. Biol. 1999, 26: 943; Garnett, Adv. Drug Deliv. Rev. 2002, 53: 171.

Compositions comprising heterologous proteins, peptides or polypeptides conjugated to a polypeptide comprising a variant IgG Fc domain are also provided.

In some aspects, a polypeptide comprising a variant IgG Fc domain is conjugated to a diagnostic or detectable agent. Such conjugates can be useful for monitoring or prognosing the development or progression of an inflammatory disorder as part of a clinical testing procedure, such as determining the efficacy of a particular therapy. Such diagnosis and detection can be accomplished by coupling a polypeptide comprising a variant IgG Fc domain to detectable substances.

In some aspects, a polypeptide comprising a variant IgG Fc domain is conjugated to a therapeutic agent. A polypeptide comprising a variant IgG Fc domain can be conjugated to a therapeutic moiety such as a cytotoxin, a therapeutic agent or a radioactive metal ion. A cytotoxin or cytotoxic agent includes any agent that is detrimental to cells. Therapeutic agents or drug moieties are not to be construed as limited to classical chemical therapeutic agents. For example, the drug moiety can be a protein or polypeptide possessing a desired biological activity. Such proteins can include, for example, a toxin, a cytokine, or a growth factor. Moreover, a polypeptide comprising a variant IgG Fc domain can be conjugated to a therapeutic moiety such as a radioactive material or a macrocyclic chelator useful for conjugating radioactive metal ions. Radioactive metals can be emitters of destructive radiation such as alpha particles for therapy or penetrating gamma radiation for diagnostic purposes. (https://world-nuclear.org/information-library/non-power-nuclear-applications/radioisotopes-research/radioisotopes-in-medicine.aspx).

An antibody comprising a variant IgG Fc domain described herein, i.e., an antibody variant, can be conjugated to a therapeutic moiety. Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56. (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al. Immunol. Rev. 62:119-58 (1982). Alternatively, antibody variant can be conjugated to a second antibody to form an antibody heteroconjugate as described by Segal in U.S. Pat. No. 4,676,980.

In some aspects, a polypeptide comprising a variant IgG Fc domain comprises one or more engineered glycoforms, i.e., a carbohydrate composition that is covalently attached to the polypeptide. Engineered glycoforms can be useful for a variety of purposes, including but not limited to reducing effector function. Engineered glycoforms can be generated by any method known to one skilled in the art, for example by using engineered or variant expression strains, by co-expression with one or more enzymes, for example DI N-acetylglucosaminyltransferase III (GnTI11), by expressing a polypeptide comprising a variant IgG Fc domain in various organisms or cell lines from various organisms, or by modifying carbohydrate(s) after a polypeptide comprising a variant IgG Fc domain has been expressed. Methods for generating engineered glycoforms are known in the art.

### Fusion Proteins

An Fc fusion protein combines an Fc domain of an immunoglobulin or fragment thereof, with a fusion partner, which in general can be any protein, polypeptide, peptide, or small molecule. The role of the non-Fc part of the Fc fusion protein, i.e., the fusion partner, is often but not always to mediate target binding, and thus is functionally analogous to the variable regions of an antibody. Accordingly, a fusion protein, i.e., a polypeptide comprising a variant IgG Fc domain and a fusion partner that specifically binds to a molecule (e.g., a cell surface receptor, chemokine, etc) is provided.

In some aspects, a fusion protein can comprise a peptide, polypeptide, protein scaffold, scFv, dsFv, diabody, Tandab, or an antibody mimetic fused to a polypeptide comprising a variant IgG Fc domain. In some aspects, a fusion protein can comprise a linker region connecting a peptide, polypeptide, protein scaffold, scFv, dsFv, diabody, Tandab, or an antibody mimetic to a polypeptide comprising a variant IgG Fc domain. The use of naturally occurring as well as artificial peptide linkers to connect polypeptides into novel linked fusion polypeptides is well known in the literature.

In some aspects, a fusion protein can combine a variant IgG Fc domain with a fusion partner which in general can be a protein, including, but not limited to, a ligand, an enzyme, the ligand portion of a receptor, an adhesion protein, or some other protein or domain.

In another aspect, a fusion protein comprises a bioactive molecule fused to a variant IgG Fc domain described herein. Bioactive molecules that can be fused to a variant IgG Fc domain described herein, but are not limited to, peptides, polypeptides, proteins, small molecules, mimetic agents, synthetic drugs, inorganic molecules, and organic molecules. In one aspect, a bioactive molecule is a polypeptide comprising at least 5, at least 10, at least 20, at least 30, at least 40, at least 50, at least 60, at least 70, at least 80, at least 90 or at least 100 contiguous amino acid residues, and is heterologous to the amino acid sequence of a variant IgG Fc domain described herein.

A fusion protein comprising a variant IgG Fc domain described herein can be fused to a marker sequence, such as but not limited to, a peptide, to facilitate purification. In some aspects, the marker amino acid sequence is a His6 tag, a “flag” tag, a hemagglutinin “HA” tag, or one of many others commercially available tags.

A variety of linkers can be used to covalent link a polypeptide comprising a variant IgG Fc domain to a fusion partner to generate a fusion protein. Alternatively, polypeptides, proteins and fusion proteins can be produced by standard recombinant DNA techniques or by protein synthetic techniques, e.g., by use of a peptide synthesizer.

### Recombinant Polypeptide Expression

The recombinant expression of a polypeptide comprising a variant IgG Fc domain, derivative, analog or fragment thereof, e.g., an antibody variant or a fusion protein comprising a variant IgG Fc domain described herein, can be accomplished through the construction of an expression vector containing a polynucleotide that encodes the polypeptide. Once a polynucleotide encoding a polypeptide comprising a variant IgG Fc domain (e.g., an antibody variant or a fusion protein) has been obtained, the vector for the production of the polypeptide can be produced by recombinant DNA technology using techniques well known in the art.

Thus, methods for preparing a protein by expressing a polynucleotide containing a nucleotide sequence encoding a polypeptide comprising a variant IgG Fc domain (e.g., an antibody variant or a fusion protein) are described herein. Methods that are well known to those skilled in the art can be used to construct expression vectors containing coding sequences and appropriate transcriptional and translational control signals. These methods include, for example, in vitro recombinant DNA techniques, synthetic techniques, and in vivo genetic recombination. Thus, replicable vectors are provided which comprise a nucleotide sequence encoding a polypeptide comprising a variant IgG Fc domain, operably linked to a promoter.

The expression vector is transferred to a host cell by conventional techniques and the transfected cells are then cultured by conventional techniques to produce a polypeptide comprising a variant IgG Fc domain. Thus, host cells are provided which contain a polynucleotide encoding a polypeptide comprising a variant IgG Fc domain, operably linked to a heterologous promoter.

A variety of host-expression vector systems can be utilized to express a polypeptide comprising a variant IgG Fc domain. Such host-expression systems represent vehicles by which the coding sequences of interest can be produced and subsequently purified, but also represent cells which can, when transformed or transfected with the appropriate nucleotide coding sequences, express a polypeptide comprising a variant IgG Fc domain in situ. These include but are not limited to microorganisms such as bacteria (e.g., E. coli and B. subtilis) transformed with recombinant bacteriophage DNA, plasmid DNA or cosmid DNA expression vectors containing a sequence or sequences encoding a polypeptide comprising a variant IgG Fc domain; yeast (e.g., Saccharomyces pichia) transformed with recombinant yeast expression vectors containing a sequence or sequences encoding a polypeptide comprising a variant IgG Fc domain; insect cell systems infected with recombinant virus expression vectors (e.g., baculovirus) containing a sequence or sequences encoding a polypeptide comprising a variant IgG Fc domain; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a sequence or sequences encoding a polypeptide comprising a variant IgG Fc domain; or mammalian cell systems (e.g., COS, CHO, BHK, 293, NSO, 3T3 cells) harboring recombinant expression constructs containing promoters derived from the genome of mammalian cells or from mammalian viruses.

A host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Eukaryotic host cells which possess the cellular machinery for proper processing of the primary transcript, glycosylation, and phosphorylation of the gene product can be used. Such mammalian host cells include but are not limited to CHO, VERY, BHK, PER.C6, HeLa, COS, MDCK, 293, 3T3, W138, BT483, Hs578T, HTB2, BT20 and T47D, NSO, CRL7030 and HsS78Bst cells.

For long-term, high-yield production of recombinant proteins, stable expression is often preferred. For example, cell lines which stably express a polypeptide comprising a variant IgG Fc domain can be engineered using methods known in the art.

Once a polypeptide comprising a variant IgG Fc domain (e.g., an antibody variant or a fusion protein) has been produced by recombinant expression, it can be purified by any method known in the art for purification of a protein, for example, by chromatography (e.g., ion exchange, affinity, particularly by affinity for the specific antigen after Protein A, and sizing column chromatography), centrifugation, differential solubility, or by any other standard technique for the purification of proteins.

### Characterization and Functional Assays

A polypeptide comprising a variant IgG Fc domain as described herein can be characterized in a variety of ways. In particular, a polypeptide comprising a variant IgG Fc domain can be assayed for the ability to specifically bind to a ligand, e.g., FcγRIIb, FcγRIIIa(158V), FcRn. Such an assay can be performed in solution (see, e.g., Houghten, Bio/Techniques 13:412-421 (1992)), on beads (see, e.g., Lam, Nature 354:82-84 (1991)), on chips (see, e.g., Fodor, Nature 364:555-556 (1993)), on bacteria (see, e.g., U.S. Pat. No. 5,223,409), on plasmids (see, e.g., Cull et al., Proc. Natl. Acad. Sci. USA 89:1865-1869 (1992)), or on phage (see, e.g., Scott and Smith, Science 249:386-390 (1990); Devlin, Science 249:404-406 (1990); Cwirla et al., Proc. Natl. Acad. Sci. USA 87:6378-6382 (1990); and Felici, J. Mol. Biol. 222:301-310 (1991)). Molecules that have been identified to specifically bind to a ligand, e.g., FcγRIIb, FcγRIIIa, FcRn can then be assayed for their affinity for the ligand.

A polypeptide comprising a variant IgG Fc domain can be assayed for specific binding to a molecule such as an antigen (e.g., cancer antigen and cross-reactivity with other antigens) or a ligand (e.g., FcγR) by any method known in the art. Immunoassays which can be used to analyze specific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, agglutination assays, complement-fixation assays, fluorescent immunoassays, protein A immunoassays, etc. Such assays are routine and well known in the art. See, e.g., Ausubel et al., eds, 1994, Current Protocols in Molecular Biology, Vol. 1, John Wiley & Sons, Inc., New York.

The binding affinity of a polypeptide comprising a variant IgG Fc domain to a molecule such as an antigen or a ligand, e.g., FcγR, and the off-rate of the interaction can be determined by competitive binding assays. The kinetic parameters of a polypeptide comprising a variant IgG Fc domain can also be determined using any surface plasmon resonance (SPR) based assays known in the art (e.g., BIAcore or ProteOn kinetic analysis). See, e.g., Mullet et al. Methods 22: 77-91 (2000); Dong et al. Rev. Mol. Biotech. 82: 303-23 (2002); Fivash et al. Curr. Opin. Biotechnol. 9: 97-101 (1998); Rich et al. Curr. Opin. Biotechnol. 11: 54-61 (2000). Additionally, any of the SPR instruments and SPR based methods for measuring protein-protein interactions described in U.S. Pat. Nos. 6,373,577; 6,289,286; 5,322,798; 5,341,215; 6,268,125 are contemplated in the methods of the present disclosure.

Fluorescence activated cell sorting (FACS), using any of the techniques known to those skilled in the art, can be used for characterizing the binding of a polypeptide comprising a variant IgG Fc domain to a molecule expressed on the cell surface (e.g., a FcγR).

A polypeptide comprising a variant IgG Fc domain can be assayed for its ability to mediate FcγR-mediated effector cell function. Examples of effector cell functions that can be assayed include, but are not limited to, antibody-dependent cell mediated cytotoxicity (ADCC), C1q binding, and complement dependent cell mediated cytotoxicity (CDC). Any cell-based or cell free assay known to those skilled in the art for determining effector cell function activity can be used (see, e.g., Perussia et al. Methods Mol. Biol. 121: 179-92 (2000); Baggiolini et al. Experientia 44: 841-8 (1998); Lehmann et al. J. Immunol. Methods 243: 229-42 (2000); Brown, Methods Cell Biol. 45: 147-64 (1994); Munn et al. J. Exp. Med. 172: 231-237 (1990); Abdul-Majid et al. Scand. J. Immunol. 55:70-81 (2002); Ding et al. Immunity 8:403-411 (1998)). In particular, a polypeptide comprising a variant IgG Fc domain can be assayed for FcγR-mediated ADCC activity in effector cells, e.g., natural killer cells, using any of the standard methods known to those skilled in the art (see, e.g., Perussia et al. Methods Mol. Biol. 121: 179-92 (2000)).

### Pharmaceutical Compositions and Methods of Administration

In another aspect, pharmaceutical compositions are provided which comprise a polypeptide comprising a variant IgG Fc domain as described herein, or a conjugate as described herein or a vector as described herein, or combinations thereof formulated together with a carrier, and a pharmaceutically acceptable excipient.

In another aspect, compositions are provided which comprise a polypeptide comprising a variant IgG Fc domain as described herein, a nucleic acid encoding a polypeptide comprising a variant IgG Fc domain as described herein, or combinations thereof formulated together with a carrier. Such compositions can include one or a combination of (e.g., two or more different) antibodies, fusion proteins, or conjugates. In some aspects, such compositions are physiologically tolerable and as such are suitable for therapeutic, prophylactic, or diagnostic administration to a subject.

In another aspect, compositions comprising a polypeptide comprising a variant IgG Fc domain (e.g., an antibody variant, a fusion protein, or a conjugate) or a nucleic acid encoding a polypeptide comprising a variant IgG Fc domain can include one or more pharmaceutically acceptable salts. Examples of suitable aqueous and nonaqueous carriers that can be employed in contemplated compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In another aspect, compositions comprising a polypeptide comprising a variant IgG Fc domain (e.g., an antibody variant, a fusion protein, or a conjugate) or a nucleic acid encoding a polypeptide comprising a variant IgG Fc domain can also contain agents such as preservatives, wetting agents, emulsifying agents and dispersing agents. Prevention of presence of microorganisms can be ensured both by sterilization procedures and by the inclusion of various antibacterial and antifungal agents, for example, paraben, chlorobutanol, phenol, sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions is contemplated. Supplementary active compounds can also be incorporated into the compositions. In some aspects, acceptable carriers include excipients approved for or considered to be safe for human and animal administration, i.e., GRAS substances (generally regarded as safe). GRAS substances are listed by the Food and Drug administration in the Code of Federal Regulations (CFR) at 21 CFR 182 and 21 CFR 184, incorporated herein by reference.

Actual dosage levels of the active ingredients in pharmaceutical compositions comprising a polypeptide comprising a variant IgG Fc domain (e.g., an antibody variant, a fusion protein, or a conjugate) or a nucleic acid encoding a polypeptide comprising a variant IgG Fc domain can be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A therapeutically effective dosage of a polypeptide comprising a variant IgG Fc domain, a nucleic acid encoding a polypeptide comprising a variant IgG Fc domain, or a pharmaceutical composition thereof results in a decrease in severity of disease symptoms, an increase in frequency and duration of disease symptom-free periods, or a prevention of impairment or disability due to the disease affliction. A therapeutically effective dose can also prevent or delays onset of disease. Accordingly, any clinical or biochemical monitoring assay can be used to determine whether a particular treatment is a therapeutically effective dose. One of ordinary skill in the art would be able to determine such amounts based on such factors as the subject's size, the severity of the subject's symptoms, and the particular composition or route of administration selected.

A composition comprising a polypeptide comprising a variant IgG Fc domain (e.g., an antibody variant, a fusion protein, or a conjugate) or a nucleic acid encoding a polypeptide comprising a variant IgG Fc domain can be administered via one or more routes of administration using one or more of a variety of methods known in the art. As will be appreciated by the skilled artisan, the route and/or mode of administration will vary depending upon the desired results. Selected routes of administration compositions comprising a polypeptide comprising a variant IgG Fc domain, a nucleic acid encoding a polypeptide comprising a variant IgG Fc domain, and pharmaceutical compositions thereof include intravenous, intramuscular, intradermal, intraperitoneal, subcutaneous, spinal or other parenteral routes of administration, for example by injection or infusion. Parenteral administration can represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, compositions comprising a polypeptide comprising a variant IgG Fc domain, a nucleic acid encoding a polypeptide comprising a variant IgG Fc domain, and pharmaceutical compositions thereof can be administered via a non-parenteral route, such as a topical, epidermal or mucosal route of administration, for example, intranasally, orally, vaginally, rectally, sublingually or topically.

### Methods of Treatment

A polypeptide comprising a variant IgG Fc domain (e.g., an antibody variant, a fusion protein, or a conjugate) or a nucleic acid encoding a polypeptide comprising a variant IgG Fc domain can be administered to an animal, in particular a mammal, specifically, a human, preferably a woman, more preferably a pregnant woman for preventing, treating, or ameliorating one or more symptoms associated with a disease, disorder, or infection.

A polypeptide comprising a variant IgG Fc domain or a nucleic acid encoding a polypeptide comprising a variant IgG Fc domain can be particularly useful for the treatment or prevention of diseases or disorders related to pregnancy.

A polypeptide comprising a variant IgG Fc domain or a nucleic acid encoding a polypeptide comprising a variant IgG Fc domain, and compositions thereof can be particularly useful for the treatment or prevention of hypertension-related conditions or pre-eclampsia/eclampsia.

A polypeptide comprising a variant IgG Fc domain or a nucleic acid encoding a polypeptide comprising a variant IgG Fc domain can be provided in pharmaceutically acceptable compositions as known in the art or as described herein. As detailed below, a polypeptide comprising a variant IgG Fc domain or a nucleic acid encoding a polypeptide comprising a variant IgG Fc domain can be used in methods of treating or preventing hypertension-related conditions or pre-eclampsia/eclampsia.

A polypeptide comprising a variant IgG Fc domain or a nucleic acid encoding a polypeptide comprising a variant IgG Fc domain, and compositions thereof can also be advantageously utilized in combination with other therapeutic agents known in the art for the treatment or prevention of hypertension-related conditions or pre-eclampsia/eclampsia. A polypeptide comprising a variant IgG Fc domain or a nucleic acid encoding a polypeptide comprising a variant IgG Fc domain, and compositions thereof can also be advantageously utilized in combination with one or more drugs used to treat a disease, disorder, or infection such as, for example anti-cancer agents, anti-inflammatory agents or anti-viral agents.

In some aspects, methods for preventing, treating, or ameliorating one or more symptoms associated with hypertension-related conditions or pre-eclampsia/eclampsia and related conditions by administering a polypeptide comprising a variant IgG Fc domain or a nucleic acid encoding a polypeptide comprising a variant IgG Fc domain are provided.

The disclosure also encompasses methods for treating or preventing hypertension-related conditions or pre-eclampsia/eclampsia in a subject comprising administering a therapeutically or prophylactically effective amount of a polypeptide comprising a variant IgG Fc domain.

In another aspect, a polypeptide comprising a variant IgG Fc domain or a nucleic acid encoding a polypeptide comprising a variant IgG Fc domain, or a conjugate and compositions thereof for use in therapy of a mammal, preferably a human, more preferably a woman, even more preferably a pregnant woman is provided and preferably for treating or preventing hypertension-related conditions or pre-eclampsia/eclampsia.

### Kits

Also provided is a pharmaceutical pack or kit comprising one or more containers filled with one or more of the pharmaceutical compositions disclosed herein. Optionally associated with such container(s) can be a notice in the form prescribed by a governmental agency regulating the manufacture, use or sale of pharmaceuticals or biological products, which notice reflects approval by the agency of manufacture, use or sale for human administration. The present disclosure provides kits that can be used in the above methods of treatment and administration. In one aspect, a kit comprises a polypeptide comprising a variant IgG Fc domain (e.g., an antibody variant, a fusion protein, or a conjugate), preferably in a purified form, in one or more containers.

### EXAMPLES

### Materials and General Methods

An overview of the nucleotide sequences encoding human immunoglobulin light and heavy chains is given in: Kabat, E. A., et al, Sequences of Proteins of Immunological Interest, 5th ed., Public Health Service, National Institutes of Health, Bethesda, MD (1991). Amino acids of antibody chains are numbered and referred to according to EU numbering as determined for the ‘EU antibody’ (Edelman, G. M., et al., Proc. Natl. Acad. Sci. USA 63 (1969) 78-85; Kabat, E. A., et al, Sequences of Proteins of Immunological Interest, 5th ed., Public Health Service, National Institutes of Health, Bethesda, MD (1991)), Specifically, the Kabat EU index numbering system (see pages 661-723) is used for the constant heavy chain domains (CH1, Hinge, CH2 and CH3).

In accordance with the present invention there may be employed conventional molecular biology, microbiology, protein expression and purification, antibody, and recombinant DNA techniques within the skill of the art. Such techniques are explained fully in the literature. See, e.g., Sambrook, J., et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ.; Nucleic Acid Hybridization, Hames & Higgins eds. (1985); Transcription And Translation, Hames & Higgins, eds. (1984); Animal Cell Culture Freshney, ed. (1986); Immobilized Cells And Enzymes, IRL Press (1986); Perbal, A Practical Guide To Molecular Cloning (1984); and Harlow and Lane. Antibodies: A Laboratory Manual (Cold Spring Harbor Laboratory Press: 1988). Antibodies are routinely expressed and purified from mammalian cells, HEK293 and CHO typically, but other expression systems such as yeast, plant cells and E. coli can also be used (Frenzel, A., et al; Expression of Recombinant Antibodies; Frontiers in Immunology: 2013, 4, Article 271: 1-20).

### Genetic Constructs and Plasmids.

Gene sequences for expression of antibody chains and other desired sequences can be codon-optimised, synthesised and sequence-confirmed by contractors such as the Geneart Services provided by Thermofisher Scientific (www.thermofisher.com/ch/en/home/life-science/cloning/gene-synthesis.html). Desired sequences for DNA synthesis are provided electronically and after codon optimisation for the appropriate expression system are synthesised and checked by DNA sequencing.

Confirmed sequences are then cloned into a suitable plasmid such as pcDNA3.4-TOPO from Thermofisher Scientific, engineered to support transient expression of target proteins in mammalian cell culture. The pcDNA3.4-TOPO incorporates features and control sequences which allow: PCR-amplified and insertion of the target gene sequence; E. coli amplification and selection for correct plasmids; plasmid purification and linearisation; transfection of mammalian cells and high level production and secretion of the target protein. In the specific instance of the pcDNA3.4-TOPO plasmid the necessary features are: WPRE (Woodchuck posttranscriptional regulatory element) downstream of the cloning site to enhance transcript expression; full-length human cytomegalovirus (CMV) immediate-early promoter/enhancer for high-level gene expression in mammalian cells—HEK 293 or CHO for example; TOPO cloning site for rapid and efficient cloning of Taq-amplified PCR products; Herpes Simplex Virus thymidine kinase polyadenylation signal for proper termination and processing of the recombinant transcript; neomycin resistance gene for selection of stable cell lines with the antibiotic geneticin; pUC origin for high copy replication and maintenance of the plasmid in E. coli; and ampicillin (bla) resistance gene for selection in E. coli. Other plasmids with similar design features can be utilised and are commonly available.

In the following Examples, the antibody with the natural sequence heavy chain (SEQ ID NO: 11) and the light chain (SEQ ID NO: 12) is designated MOm301 and also known as WBP70323_1 and BB301. The antibody with the substitutions 1253A+H310A+H435A/L234F+L235E+P331S in the heavy chain (SEQ ID NO: 13) and the light chain (SEQ ID NO: 12) is designated M n303 and also known as WBP70323_2 and BB303.

Data from the Examples below is shown in the figures as follows:

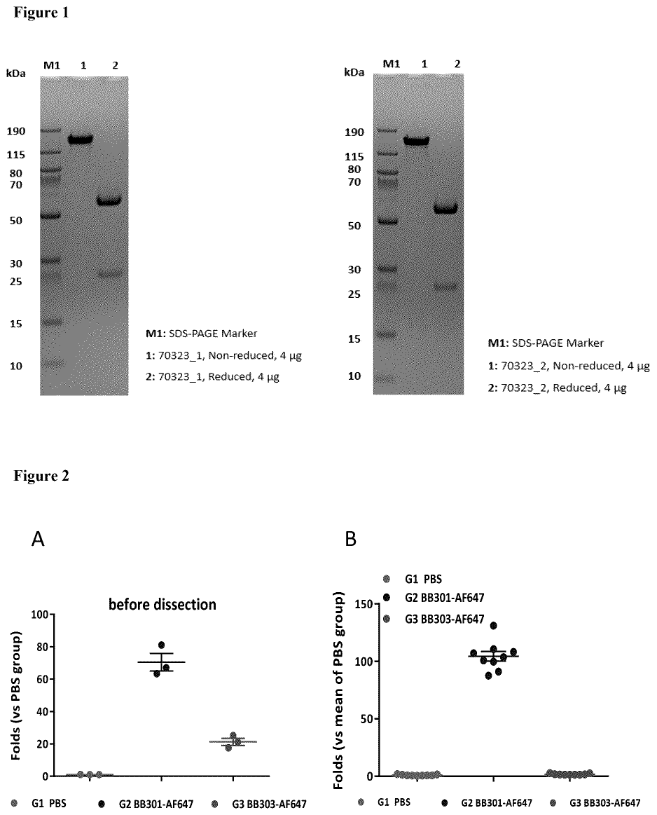

FIG. 1. Gel electrophoresis of purified antibodies WBP70323_1 (MOm301) and WBP70323_2 (MOm303

FIG. 2 Fold changes of total fluorescence signal of pregnant mice and foetuses

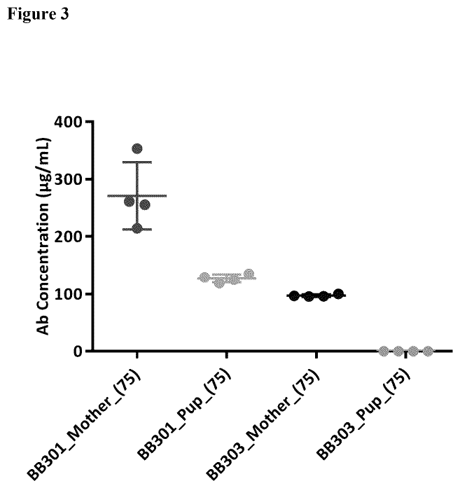

FIG. 3 Plasma concentrations of BB301 and BB303 in pregnant mice and foetuses 24 hours after dosing.

### Example 1. Production of MOm301 and MOm303 by Transient Expression in CHO Cells

Codon-optimised DNA sequences were synthesized and cloned into expression vectors designed to express approximately equivalent molar quantities of the respective heavy and light chains by transient expression in CHO K1 cells. Pilot expressions to confirm the functionality of expression vectors were performed. The vectors were then replicated and purified in sufficient quantities for transient expression in cultures of 2 litres. CHO-K1 host cells were thawed and cultured in BM001H medium (WuXi Bio internal Cat. Number) containing 4 mM Glutamine (J. T. Baker, 2078-06) and 1% HT Supplement (Gibco, 11067-030) in preparation for transfections. Separate cultures were transfected with a mixture of equal masses of the light and heavy chain vectors for WBP70323_1 (vectors PWX4.1-HC-70323_1 and PWX4.1-LC-70323_1) and for WBP70323_2 (PWX4.1-HC-70323_2 and PWX4.1-LC-70323_2). Cells were expanded in BM022H media (WuXi Bio internal Cat. Number) containing 6 mM Glutamine (ITW Reagents A1420,1000) and 1% HT Supplement (Gibco, 11067-030) and the same media was used for transfection and antibody production. The feeding media used to support production were FM020a (Hyclone-SH31026.01) and FM020b (Hyclone-SH31027.01). Transient transfections were performed for WBP70323_1 and WBP70323_2 in individual 5L shake flasks by mixing the CHO-K1 host cells with polyethyleneimine (PEI, BIOHUB) and plasmid DNA. The host cells were seeded at 1.8-2.0×10⁶ cells/mL in BM024H medium 96 hours before transfection. Cells were counted for cell density using a Vi-CELL counter, and diluted with pre-warmed BM022H prior to transfection. The diluted host cells were incubated in a Kuhner shaker (36.5° C., 6% CO₂, 150 rpm, 50 mm diameter) before use. Vector DNA, 1.25 mg each of the appropriate heavy and light chain vectors for the respective antibodies, was added to the diluted host cells, followed by 12 mg of PEI. The transfected cultures were incubated with shaking for 2 hours before the addition of feeding media and continued incubation for 4 days at which time the antibodies were harvested. On the harvest day, cell cultures were clarified by centrifugation at 10,000×g for 40 mins, followed by sterile filtration through a 0.22 μm filter. WBP70323_1 was captured from the supernatant and purified by protein A chromatography using MabSelect SuRe (Cytiva, 17543803). WBP70323_2 was captured and purified using Capto™ L (Cytiva, 17547802).

Chromatography was performed using an AKTA Pure M150 system. MabSelect SuRe was packed in a 5.0 cm diameter column with a 7.3 cm bed height with a column volume of 140 ml. Capto™ L was packed in a 5.0 cm diameter column with a 7.7 cm bed height with a column volume is 150 ml. All runs were conducted in bind-elute mode and the bound protein was eluted by low pH. Eluated fractions for each molecule were tested for concentration and purity. The collected fractions of affinity-purified WBP70323_1 and, separately, WBP70323_2, pooled fractions were further purified by SEC chromatography using Superdex200 (Cytiva, 17104302). Superdex200 resin was packed in a 5.0 cm diameter column with a 87.6 cm bed height and the packed volume was 1720 mL. The fractions containing the individual purified antibodies were concentrated to about 20 mg/mL in 20 mM Histidine Acetate, 150 mM NaCl, pH 5.5 buffer using 30 kDa Amicon Ultra-15 mL Centrifugal Filter Units (Millipore UFC903096) and clarified by sterile filtration through 0.22 μm filters. The antibodies were measured for protein content, endotoxin and analysed by SEC, gel electrophoresis and mass spectrometry. The SDS-PAGE profiles under non-reducing and reducing conditions are shown in FIG. 1. The masses for the non-reduced antibodies are consistent with the known masses for intact IgG antibodies as are the masses for the individual heavy and light chains after separation under reducing conditions.

### Example 2. Binding Characteristics to VEGFR-1, FcγRIIb, FcγRIIIa and FcRn

Surface plasmon residence (SPR) is commonly used to measure binding affinities of antibodies to ligands and Fc receptors. SPR measures the change in refractive index caused by mass differences due to the binding, or unbinding, of a protein to a gold coated sensor. The Biocore™ 8k (Cytivia) is widely used for measuring antibody binding interactions: real-time increases and decreases in mass, reported as resonance units (RU), are used to measure the association (kₐ) and dissociation (k_{d}) rate constants of binding and determine the equilibrium constant KD. Multicycle kinetics analysis was performed using a Biocore™ instrument running Biocore™ Insight Software in order to measure antibody binding to the ligand, VEGFR-1, and to Fc receptors.

Binding measurements were conducted to evaluate the binding of BB301 (MOm301, WBP70323_1) and BB303 (MOm303, WBP70323_2) to the target ligand, VEGFR-1, the Fcγ receptors IIb and IIIa and FcRn at two different pH values. In parallel a third sample, BB301 modified with the dye Alexa Fluor™ 647 and identified as BB301-AF647, was analysed. This analysis was performed to investigate if the dye modification, as described in Example 3, had any effect on either the binding to the target ligand, VEGFR-1, the Fcγ receptors IIb or IIIa or the FcRn receptor compared to the unmodified BB301 antibody.

Binding to VEGFR-1. Activator solution was prepared by mixing 400 mM 1-ethyl-3-(3-dimethylaminopropyl) carbodiimide (EDC) and 100 mM N-Hydroxy Succinimide (NHS) immediately prior to injection and activation of a CM5 sensor chip at a flow rate of 10 μL/min for 420s. VEGFR1 (H.pro.1 His: Sinobiologics, 10136-H08H1) at a concentration of 0.8 μg/mL in 10 mM NaAc (pH 5.0) was then injected to flow cell 2 (Fc2) for 60s at a flow rate of lOL/min. The reference channel Fcl was blocked and not exposed. The chip was deactivated by IM ethanolamine-HCl for 420s at a flow rate of 10 μL/min. Six concentrations (0.625, 1.25, 2.5, 5, 10, 20 nM) of the analytes BB301, BB303 or BB301-AF647 in running buffer (1×HBS-EP+; 0.1 M HEPES, 1.5 M NaCl, 0.03 M EDTA and 0.5% v/v Surfactant P20; Cytiva BR100669) were injected at a flow rate of 30 μL/min for an association phase of 240s followed by a dissociation phase of 3600s. The chip was regenerated by the injection of 10 mM glycine (pH 1.5) buffer following each dissociation phase.

The sensorgrams for the reference channel and buffer channels were subtracted from the test sensorgrams and experimental data fitted to a 1:1 binding model. A molecular weight of 146,508 Da was used to calculate the molar concentration of the analytes BB301 and BB301-AF647. A molecular weight of 146,239 Da was used to calculate the molar concentration of the analyte BB303.

TABLE 1
Affinity of BB301, BB303 and BB301-AF647 for human VEGFR1

| | | | | | Immobilized | Rmax |
|---|---|---|---|---|---|---|
| Ligand | Analyte | kₐ (1/Ms) | k_{d} (1/s) | K_{D} (M) | level (RU) | (RU) |
| Human | BB301 | 7.99E+05 | 1.31E−05 | 1.64E−11 | 64 | 18.8 |
| VEGFR1 | BB303 | 7.94E+05 | 1.21E−05 | 1.52E−11 | 62.1 | 17.5 |
| | BB301-AF647 | 7.05E+05 | 1.36E−05 | 1.93E−11 | 65.3 | 17.9 |

The dissociation constants for BB301 and BB303 were 1.64×10⁻¹¹ M and 1.52×10⁻¹¹ M respectively. The results confirmed that the amino acid substitutions introduced into the Fc region of BB303 have no effect on the binding affinity for VEGFR-1. The dissociation constant for BB301-AF647 is 1.93×10⁻¹¹ M which is no different from BB301 within the variation of the method. The result confirms that the modification of BB301 with Alexa Fluor™ 647 has no effect on VEGFR-1 binding.

Binding to Fcγ receptor IIb. The CM5 sensor chip was activated for 420s with activator solution at a flow rate of 10 μL/min. Anti-his tag antibody (THE™, Genescript, A00186-100), at a concentration of 30 μg/mL in 10 mM NaAc pH 4.5, was then injected at a flow rate of 30 μL/min for 400s. The chip was deactivated with 1M ethanolamine-HCl (Cytiva) at a flow rate of 10 μL/min for 420s. His-tagged FcγRIIb (AcrobioSystems, CDB-H5228) at a concentration of 0.7 μg/mL in running buffer (1×HBS-EP+), was injected to Fc2 at a flow rate of 10 μL/min for 30s. Eight concentrations (160, 320, 640, 1280, 2560, 5120, 10240, 20480 nM) of analyte BB301, BB303 or BB301-AF647 in running buffer were injected at a flow rate of 30 μL/min during an association phase of 60s, followed by 90s dissociation phase. Glycine (10 mM at pH 1.5) regeneration buffer was injected to the flow cell following every dissociation phase.

The sensorgrams for reference channel and buffer channel were subtracted from the test sensorgrams. The experimental data were fitted by steady state affinity model. A molecular weight of 146,508 Da was used to calculate the molar concentration of analytes BB301 and BB301-AF647. A molecular weight of 146,239 Da was used to calculate the molar concentration of analyte BB303.

TABLE 2
Affinity of BB301, BB303 and BB301-AF647 for FcγRIIb

| Ligand | Analyte | K_{D} (M) | Capture level (RU) | Rmax(RU) |
|---|---|---|---|---|
| FcγRIIb | BB301 | 4.10E−06 | 160.9 | 502.4 |
| | BB303 | 1.17E−05 | 166.6 | 275 |
| | BB301-AF647 | 4.28E−06 | 158.7 | 502.6 |

The dissociation constants for BB301 and BB303 were 4.1×10⁻⁶M and 1.17×10⁻⁵M respectively. The results demonstrate that the amino acid substitutions introduced into the Fc region of BB303 have reduced the dissociation constant, which is a measure of affinity, by three-fold compared to the unmodified antibody. The dissociation constant for BB301-AF647 is 4.28×10⁻⁶M which is highly similar to BB301, within the variation of the method. The result confirms that the modification of BB301 with Alexa Fluor™ 647 has no effect on Fcγ receptor IIb binding.

### Binding to FcγRIIIa (V176)

The CM5 sensor chip was activated for 420s with activator solution at a flow rate of 10 μL/min. THE™ anti-his tag antibody, at 30 μg/mL in 10 mM NaAc (pH 4.5), was then injected for 400s at a flow rate of 30 μL/min. The chip was deactivated by 1M ethanolamine-HCl (Cytiva) at a flow rate of 10 μL/min for 420s. His-tagged FcγRIIIa (AcrobioSystems, CD8-H52H4), 0.5 μg/mL in running buffer (1×HBS-EP+), was injected to Fc2 at a flow rate of 10 μL/min for 30s. Individual injections of BB301 and BB301-AF647 at concentrations of 5, 10, 20, 40, 80, 160, 320 and 640 nM and BB303 at concentrations of 80, 160, 320, 640, 1280, 2560, 5120, 10240 nM in running buffer were made at a flow rate of 30 μL/min with an association phase of 300s, followed by 300s dissociation. Glycine (10 mM at pH 1.5) regeneration buffer was injected to the flow cell following injection.

TABLE 3
Affinity of BB301, BB303 and BB301-AF647 to FcγRIIIa (V176)

| | | | Capture level | |
|---|---|---|---|---|
| | Analyte | K_{D} (M) | (RU) | Rmax(RU) |
| FcγRIIIa | BB301 | 9.74E−08 | 85.3 | 311.6 |
| (V176) | BB303 | 3.90E−06 | 85.4 | 202.7 |
| | BB301-AF647 | 9.86E−08 | 84.3 | 309.5 |

The dissociation constants for BB301 and BB303 are 9.74×10⁻⁸M and 3.9×10⁻⁶M respectively. The results demonstrate that the amino acid substitutions introduced into the Fc region of BB303 have reduced the dissociation constant, which is a measure of the affinity, by forty-fold compared to the unmodified antibody. The dissociation constant for BB301-AF647 is 9.86×10⁻⁸ M which is highly similar to BB301, within the variation of the method. The result confirms that the modification of BB301 with Alexa Fluor™ 647 has no effect on Fcγ receptor IIIa binding.

### Binding to Human FcRn at pH 6.0 and pH 7.4

In preparation, human FcRn (AcrobioSystems, FCM-H5286) was buffer-exchanged into pH 6.0 phosphate-buffered saline Tween (PBST pH6.0; 50 mM Na₂HPO₄/NaH₂PO₄, 150 mM NaCl, 0.05% Tween-20, pH 6.0) or pH 7.4 PBST (50 mM Na₂HPO₄/NaH₂PO₄, 150 mM NaCl, 0.05% Tween-20, pH 7.4) running buffer using a desalting column (Zeba Spin Desalting Columns, 7K MWCO, 0.5 mL; Thermofisher, Pierce-89882). The concentrations were determined using a NanoDrop 2000 spectrophotometer.

The sensor chip activator was prepared by mixing 400 mM EDC and 100 mM NHS immediately prior to injection. CM5 sensor chips were activated for 420s at a flow rate of 10 μL/min with the mixture. In separate experiments using dedicated chips, BB301, BB303 or BB301-AF647 at a concentration of 10 μg/mL in 10 mM NaAc (pH 5.5), were injected to Fc2 for 60s at a flow rate of 10 μL/min. Chips were deactivated by 1M ethanolamine-HCl for 420s at a flow rate of 10 μL/min. In individual experiments the binding characteristics at both pH6.0 and pH7.4 were analyzed for BB301, BB303 or BB301-AF647. Eight concentrations of human FcRn analyte (46.9, 93.7, 187.5, 375, 750, 1500, 3000, 6000 nM) in running buffer (PBST, pH 6.0 or 7.4) were injected to Fc1 and Fc2 at a flow rate of 30 μL/min for an association phase of 60s, followed by 90s dissociation for each pH and for each antibody. Regeneration buffer, PBS (50 mM Na₂HPO₄/NaH₂PO₄, 150 mM NaCl, pH 7.4), was injected to flow cells following every dissociation phase.

The sensorgrams for the reference channel and buffer channels were subtracted from the test sensorgrams. The experimental data were fitted by steady state affinity model. A molecular weight of 45 kDa was used to calculate the molar concentration of human FcRn.

TABLE 4
Affinity of BB301, BB303 and BB301-AF647 to human FcRn

| | | | Immobilized | |
|---|---|---|---|---|
| Analyte | Ligand | K_{D} (M) | level (RU) | Rmax (RU) |
| Human | BB301 | 2.06E−06 | 973.3 | 433 |
| FcRn at | BB303 | No or weak binding | 927.9 | Not detectable |
| pH 6.0 | BB301- | 1.66E−06 | 888.6 | 357.2 |
| | AF647 | | | |
| Human | BB301 | No or weak binding | 973.3 | Not detectable |
| FcRn at | BB303 | | 927.9 | Not detectable |
| pH 7.4 | BB301- | | 888.6 | Not detectable |
| | AF647 | | | |

The dissociation constants for BB301 at pH 6.0 was 2.06×10⁻⁶M whereas the binding of BB303 was not detectable. The results demonstrate that the amino acid substitutions introduced into the Fc region of BB303 have greatly diminished binding to the FcRn receptor. Binding to FcRn for both BB301 and BB303 was not detectable at pH 7.4.

At pH 6.0 the dissociation constant for BB301-AF647 is 1.66×10⁻⁶M which is highly similar to BB301, within the variation of the method. At pH 7.4 binding for BB301-AF647 is also not detectable.

### Example 3. Fluorescent Labelling of Antibodies

BB301 stock solution containing 16 mg protein was diluted and distributed between three Amicon Ultra-0.5 ml units, concentrated by centrifugation, recovered by centrifugation of the inverted units, pooled and made up to a final volume of 450 μL with 1×borate buffer. Three further rounds of centrifugation were performed to replace the original buffer and the final concentrate was diluted to 8 mL with 1×borate buffer. For labelling, forty microlitres of Alexa Fluor™ 647 NHS Ester was added, mixed and incubated for two hours at room temperature. The free dye was removed by dialysis, the solution diluted and the concentrations of antibody and dye measured using a Nanodrop spectrophotometer (Thermofisher). The protein concentration for AF647-BB301, measured at 280 nm using an extinction coefficient of 220762 cm⁻¹M⁻¹, was 2.04 mg/mL (13.9 uM) and the concentration of AF647, measured at 650 nm using an extinction coefficient of 239000 cm⁻¹M⁻¹, was 32.2 μM. The final yield of AF647-BB301 was 14.0 mg in a volume of 6.9 mL and a ratio of dye-to-protein of 2.3 to 1.

BB303 was modified by the same method adapted to accommodate the initial concentration of the antibody. The protein concentration, after modification, dialysis and dilution for AF647-BB303, measured at 280 nm using an extinction coefficient of 221215 cm⁻¹M⁻¹, was 2.15 mg/mL (14.7 μM) and the concentration of AF647, measured at 650 nm using an extinction coefficient of 239,000 cm⁻¹M⁻¹, was 33.1p M. The final yield of AF647-BB303 was 14.6 mg in a volume of 6.8 mL and a ratio of dye-to-protein of 2.3 to 1 mol/mol.

Labelled antibodies were stored at 2-8° C. in the dark and are stable for at least one month under these conditions.

In Example 2, SPR analysis of AF647-BB301 demonstrates that the dye-modified antibody has the same binding characteristics for VEGFR-1, FcγIIb, FcγIIIa and FcRn as the unmodified BB301 antibody. The labelled antibodies AF647-BB301 and AF647-BB303 have the same degree of modification, in terms of mols of dye per mol of antibody, which means that AF647-BB303 is expected to have the same binding characteristics as unmodified BB303.

### Example 4 Maternofetal Transfer with Fluorescent-Labelled Antibodies

The inhibition of maternofetal transfer by the introduction of mutations to greatly reduce or abolish receptor binding affinities was demonstrated by comparing the maternofetal transfer of labelled MOm301 (AF647-BB301), without modifications to receptor binding, with that of labelled MOm303 (AF647-BB303) which includes the modifications 1253A+H310A+H435A/L234F+L235E+P331S.

BALB/c male and female mice were housed under SPF conditions with appropriate regard for welfare and following all local regulations. Males were mated at 18 weeks and females at 16 weeks. The morning after mating pregnant females were identified by the presence of a viscous vaginal plug. Such females were designated as gestational day 0.5 (D0.5) and dosing was performed on D16.5. Pregnant mice were assigned to three groups of three based on body weight. The first group was administered phosphate-buffered saline (PBS) only, the second group received 30 mg/kg of AF647-BB301 and the third 30 mg/kg of AF647-BB303. Twenty-four hours after dosing the mice were anaesthetized with isoflurane and then subject to fluorescence imaging using an IVIS® Spectrum (Revvity, Inc.). Following imaging the pregnant mice were euthanized under anaesthesia and the foetuses dissected away from the placentas and surrounding tissue prior to imaging. Total fluorescence efficiency was averaged across three foetuses for each mouse to control for variation between foetuses. Fluorescence fold-change was calculated compared to the PBS controls. The laser excitation filter was set to 640 nm and emission filter was set to 680 nm.

The average fold change in fluorescence for pregnant females and foetuses was measured over the PBS control (FIG. 2). The females administered AF647-BB301 exhibited a 70.4 fold-increase in fluorescence; females administered AF647-BB303 exhibited a fold-increase of 21.2. The lower fold-increase for AF647-BB303 is consistent with the increase clearance due to impairment of binding to FcRn and is confirmed by the pharmacokinetic analysis of Example XXX. The foetuses of the mice administered AF647-BB301 exhibited a 104.3 fold-increase in fluorescence; foetuses of the mice administered AF647-BB303 had only a 1.74 fold-increase which is not statistically different compared to the PBS control.

In conclusion, a comparison between fluorescently-labelled BB301 (MOm301) and BB303 (MOm303) demonstrates that the amino acid substitutions I253A+H310A+H435A/L234F+L235E+P331S lead to a reduction in materno-fetal transfer of at least 98.3% (100 minus (1.74 divided by 104.3, multiplied by 100)).

### Example 5 Pharmacokinetics of MOm301 and MOm303 in BALB/c Mice

BALB/c female mice were housed under SPF conditions with appropriate concern for welfare and following all local regulations. Female mice between 8-10 weeks old were randomly assigned to four groups of five. Groups 1 and 2 were dosed with 10 mg/kg of BB301 (MOm301) and groups 3 and 4 were dosed with 10 mg/kg of BB303 (MOm303). At designated timepoints blood was collected by ocular venous puncture. Mice were fully anesthetized with isoflurane prior to collection: whole blood was collected in 1.5 mL disposable anticoagulant tubes, centrifuged at 8000 rpm for 5 minutes at 4° C., and the plasma supernatant collected for analysis by ELISA. The collection timings were: group 1, 1 h, 24 h, 72 h, 168 h; group 2, 6 h, 48 h, 120 h, 168 h; group 3, 1 h, 6 h, 24 h, 72 h and group 4, 3 h, 8 h, 48 h, 96 h.

The plasma concentrations of antibodies were measured by ELISA. Each sample was tested in duplicate against a standard curve. Briefly, Goat anti-human IgG-F(ab′)2 antibody (Bethyl Laboratories, A80-249A) was coated at 1 μg/mL on 96-well ELISA plates (100 μL per well) at 37° C. for 2.5 hours. The plates were then washed once with PBST (300 μL per well) and blocked with 2% BSA (bovine serum albumin, 200 μL per well) at ambient temperature for 2 hours. After washing three times with PBST (300 μL per well), serial dilutions of standards or plasma samples in 2% BSA were added to the plates (100 μL per well) and incubated at ambient temperature for one hour. After washing three times with PBST (300 μL per well), Goat anti-Human Ig Fab-HRP antibody (Southern Biotech, 2085-05) was added to the plates (100 μL per well) at 0.09 μg/mL and incubated at ambient temperature for one hour. After washing three times with PBST (300 μL per well), tetramethyl benzidine substrate was added to the plates for colour development for 8 minutes (100 μL per well) before the reaction was stopped by adding 2 M HCl (100 μL per well). The absorbance at 450 nm and 540 nm was determined using a microplate spectrophotometer (SpectraMax® M5e). A standard curve was generated based on the standard samples using SoftMax Pro software.

TABLE 5
Pharmacokinetics of BB301 and BB303 in female mice.

| Compound | BB301 | BB303 |
|---|---|---|
| t_{1/2} (h) | 380 | 17.0 |
| Cₘₐₓ (μg/mL) | 165 | 146 |
| AUC₀₋ₜ (h*μg/mL) | 12686 | 2429 |
| Cl_obs (mL/day/kg) | 5.25 | 97.4 |
| MRTINF_obs (h) | 539 | 19.6 |
| Vss_obs (mL/kg) | 118 | 79.7 |

In conclusion, BB301 (MOm301) exhibits a half-life of 380 hours in female BALB/c mice and BB303 (MOm303) shows that the amino acid substitutions I253A+H310A+H435A/L234F+L235E+P331S lead to a reduction half-life to 17 hours.

### Example 6 Maternofetal Transfer Measured by Enzyme-Linked Immunoassay (ELISA)

The inhibition of maternofetal transfer by the introduction of mutations to greatly reduce or abolish receptor binding affinities was demonstrated by comparing the maternofetal transfer of BB301 (MOm301), without modifications to receptor binding, with that of BB303 (MOm303) which includes the modifications I253A+H310A+H435A/L234F+L235E+P331S.

BALB/c male and female mice were housed under SPF conditions with appropriate concern for welfare and following all local regulations. Males were mated at 18 weeks and females at 16 weeks. The morning after mating pregnant females were identified by the presence of a viscous vaginal plug. These females were designated as gestational day 0.5 (D0.5) and dosing was performed on D16.5. Pregnant mice were assigned to three groups of four based on body weight. The first group was administered 70 mg/kg BB301, the second group received 70 mg/kg of BB303 and the third phosphate-buffered-saline (PBS) as a control. Analysis of samples for pregnant female and foetuses was performed 24 hours after administration of BB301 and BB303.

Pregnant mice were fully anesthetized with isoflurane prior to blood collection from the ocular venous plexus. Blood was collected from foetuses after decapitation and then pooled. Whole blood was collected in 1.5 mL disposable anticoagulant tubes, centrifuged at 8000 rpm for 5 minutes at 4° C., and the plasma supernatant collected for analysis by ELISA according to the method described in Example 5.

FIG. 3 shows the plasma levels of BB301 and BB303 in pregnant mice and foetuses. After 24 hours the BB301 plasma levels for the pregnant female was 271 (SD 58.7); the level for the foetus was 127 (SD 6.73); for BB303 the plasma levels for the pregnant female was 97.0 (SD 2.10); the level for the foetus was 0.27 (SD 0.02).

In conclusion, based on analysis by quantitative ELISA, a comparison between BB301 (MOm301) and BB303 (MOm303) demonstrates that the amino acid substitutions I253A+H310A+H435A/L234F+L235E+P331S lead to a reduction in materno-fetal transfer of at least 99.8% (100 minus (0.27 divided by 127 multiplied by 100)).

TABLE 6
Description of sequences

| | |
|---|---|
| SEQ ID NO: 1 | P01857 (uniprot.org) |
| | Immunoglobulin heavy chain constant region gamma 1; G1m1,17 allotype |
| | Sequence starts at 118 by the Kabat EU index numbering system |
| SEQ ID NO: 2 | P01859 |
| | Immunoglobulin heavy chain constant region gamma 2 |
| | Sequence starts at 118 by the Kabat EU index numbering system |
| SEQ ID NO: 3 | P01860 |
| | Immunoglobulin heavy chain constant region gamma 3 |
| | Sequence starts at 118 by the Kabat EU index numbering system |
| SEQ ID NO: 4 | P01861 |
| | Immunoglobulin heavy chain constant region gamma 4 |
| | Sequence starts at 118 by the Kabat EU index numbering system |
| SEQ ID NO: 5 | Immunoglobulin heavy chain constant region gamma 1; G1m3 allotype |
| | MOm301 sequence of heavy chain not including variable region (1-117) |
| | Sequence starts at 118 by the Kabat EU index numbering system |
| SEQ ID NO: 6 | SEQ ID NO: 5 with mutations |
| | I253A_H310A_H435A\|L234A_L235A_P331S |
| | Sequence starts at 118 by the Kabat EU index numbering system |
| SEQ ID NO: 7 | SEQ ID NO: 5 with mutations H310A_H435Q\|L234A_L235A_P331S |
| | Sequence starts at 118 by the Kabat EU index numbering system |
| SEQ ID NO: 8 | SEQ ID NO: 5 with I253A_H310A_H435A\|L234F_L235E_P331S# |
| | MOm303 sequence of heavy chain not including variable region (1-117) |
| | Sequence starts at 118 by the Kabat EU index numbering system |
| SEQ ID NO: 9 | SEQ ID NO: 5 with H310A_H435Q\|L234F_L235E_P331S |
| | Sequence starts at 118 by the Kabat EU index numbering system |
| SEQ ID NO: 10 | MOm301 and MOm303 Heavy chain variable region 126 amino acids |
| SEQ ID NO: 11 | MOm301 Variable plus constant heavy chain sequences |
| | Combines SEQ ID NO: 10 and SEQ ID NO: 5 |
| SEQ ID NO: 12 | MOm301 and MOm303 Kappa light chain |
| SEQ ID NO: 13 | MOm303 Full sequence of heavy chain including the substitutions |
| | I253A_H310A_H435A\|L234F_L235E_P331S |
| | Combines SEQ ID NO: 10 + SEQ ID NO: 8 |
| SEQ ID NO: 14 | Immunoglobulin G1 with substitution N297A - Full sequence of heavy |
| | chain |
| SEQ ID NO: 15 | Immunoglobulin G4 - Full sequence of heavy chain |
| SEQ ID NO: 16 | Immunoglobulin G4 with substitution S228P - Full sequence of heavy |
| | chain |
| SEQ ID NO: 17 | Immunoglobulin G4 with substitutions S228P_L235E - Full sequence of |
| | heavy chain |
| SEQ ID NO: 18 | Immunoglobulin G2 |
| SEQ ID NO: 19 | Immunoglobulin G2/4 hybrid sequence |

TABLE 7
List of sequences identified in the Description and Claims. Mutations
and substitutions are denoted in bold and underline type.

| | |
|---|---|
| SEQ ID | ASTKGPSVFPLAPSSKSTSGGTAALGCLVKDYFPEPVTVSWNSGALTSGVHTFPAVLQS |
| NO: 1 | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKENWYVDGVEVHNAKTKPREE |
| | QYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| SEQ ID | ASTKGPSVFPLAPCSRSTSESTAALGCLVKDYFPEPVTVSWNSGALTSGVHTFPAVLQS |
| NO: 2 | SGLYSLSSVVTVPSSNFGTQTYTCNVDHKPSNTKVDKTVERKCCVECPPCPAPPVAGPS |
| | VFLFPPKPKDTLMISRTPEVTCVVVDVSHEDPEVQFNWYVDGVEVHNAKTKPREEQENS |
| | TFRVVSVLTVVHQDWLNGKEYKCKVSNKGLPAPIEKTISKTKGQPREPQVYTLPPSREE |
| | MTKNQVSLTCLVKGFYPSDISVEWESNGQPENNYKTTPPMLDSDGSFFLYSKLTVDKSR |
| | WQQGNVFSCSVMHEALHNHYTQKSLSLSPGK |
| SEQ ID | ASTKGPSVFPLAPCSRSTSGGTAALGCLVKDYFPEPVTVSWNSGALTSGVHTFPAVLQS |
| NO: 3 | SGLYSLSSVVTVPSSSLGTQTYTCNVNHKPSNTKVDKRVELKTPLGDTTHTCPRCPEPK |
| | SCDTPPPCPRCPEPKSCDTPPPCPRCPEPKSCDTPPPCPRCPAPELLGGPSVELFPPKP |
| | KDTLMISRTPEVTCVVVDVSHEDPEVQFKWYVDGVEVHNAKTKPREEQYNSTERVVSVL |
| | TVLHQDWLNGKEYKCKVSNKALPAPIEKTISKTKGQPREPQVYTLPPSREEMTKNQVSL |
| | TCLVKGFYPSDIAVEWESSGQPENNYNTTPPMLDSDGSFFLYSKLTVDKSRWQQGNIFS |
| | CSVMHEALHNRFTQKSLSLSPGK |
| SEQ ID | ASTKGPSVFPLAPCSRSTSESTAALGCLVKDYFPEPVTVSWNSGALTSGVHTFPAVLQS |
| NO: 4 | SGLYSLSSVVTVPSSSLGTKTYTCNVDHKPSNTKVDKRVESKYGPPCPSCPAPEFLGGP |
| | SVFLFPPKPKDTLMISRTPEVTCVVVDVSQEDPEVQFNWYVDGVEVHNAKTKPREEQEN |
| | STYRVVSVLTVLHQDWLNGKEYKCKVSNKGLPSSIEKTISKAKGQPREPQVYTLPPSQE |
| | EMTKNQVSLTCLVKGFYPSDIAVEWESNGQPENNYKTTPPVLDSDGSFFLYSRLTVDKS |
| | RWQEGNVFSCSVMHEALHNHYTQKSLSLSLGK |
| SEQ ID | ASTKGPSVFPLAPSSKSTSGGTAALGCLVKDYFPEPVTVSWNSGALTSGVHTFPAVLQS |
| NO: 5 | SGLYSLSSVVTVPSSSLGTQTYICNVNHKPSNTKVDKRVEPKSCDKTHTCPPCPAPELL |
| | GGPSVFLFPPKPKDTLMISRTPEVTCVVVDVSHEDPEVKENWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| SEQ ID | ASTKGPSVFPLAPSSKSTSGGTAALGCLVKDYFPEPVTVSWNSGALTSGVHTFPAVLQS |
| NO: 6 | SGLYSLSSVVTVPSSSLGTQTYICNVNHKPSNTKVDKRVEPKSCDKTHTCPPCPAPEAA |
| | GGPSVFLFPPKPKDTLMASRTPEVTCVVVDVSHEDPEVKENWYVDGVEVHNAKTKPREE |
| | QYNSTYRVVSVLTVLAQDWLNGKEYKCKVSNKALPASIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNAYTQKSLSLSPGK |
| SEQ ID | ASTKGPSVFPLAPSSKSTSGGTAALGCLVKDYFPEPVTVSWNSGALTSGVHTFPAVLQS |
| NO: 7 | SGLYSLSSVVTVPSSSLGTQTYICNVNHKPSNTKVDKRVEPKSCDKTHTCPPCPAPEAA |
| | GGPSVFLFPPKPKDTLMISRTPEVTCVVVDVSHEDPEVKENWYVDGVEVHNAKTKPREE |
| | QYNSTYRVVSVLTVLAQDWLNGKEYKCKVSNKALPASIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNQYTQKSLSLSPGK |
| SEQ ID | ASTKGPSVFPLAPSSKSTSGGTAALGCLVKDYFPEPVTVSWNSGALTSGVHTFPAVLQS |
| NO: 8 | SGLYSLSSVVTVPSSSLGTQTYICNVNHKPSNTKVDKRVEPKSCDKTHTCPPCPAPEFE |
| | GGPSVFLFPPKPKDTLMASRTPEVTCVVVDVSHEDPEVKENWYVDGVEVHNAKTKPREE |
| | QYNSTYRVVSVLTVLAQDWLNGKEYKCKVSNKALPASIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNAYTQKSLSLSPGK |
| SEQ ID | ASTKGPSVFPLAPSSKSTSGGTAALGCLVKDYFPEPVTVSWNSGALTSGVHTFPAVLQS |
| NO: 9 | SGLYSLSSVVTVPSSSLGTQTYICNVNHKPSNTKVDKRVEPKSCDKTHTCPPCPAPEFE |
| | GGPSVFLFPPKPKDTLMISRTPEVTCVVVDVSHEDPEVKENWYVDGVEVHNAKTKPREE |
| | QYNSTYRVVSVLTVLAQDWLNGKEYKCKVSNKALPASIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNQYTQKSLSLSPGK |
| SEQ ID | QAQVVESGGGVVQSGRSLRLSCAASGFAFSSYGMHWVRQAPGKGLEWVAVIWYDGSNKY |
| NO: 10 | YADSVRGRFTISRDNSENTLYLQMNSLRAEDTAVYYCARDHYGSGVHHYFYYGLDVWGQ |
| | GTTVTVSS |
| SEQ ID | QAQVVESGGGVVQSGRSLRLSCAASGFAFSSYGMHWVRQAPGKGLEWVAVIWYDGSNKY |
| NO: 11 | YADSVRGRFTISRDNSENTLYLQMNSLRAEDTAVYYCARDHYGSGVHHYFYYGLDVWGQ |
| | GTTVTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKRVEPKSCDKTHTCP |
| | PCPAPELLGGPSVELFPPKPKDTLMISRTPEVTCVVVDVSHEDPEVKENWYVDGVEVHN |
| | AKTKPREEQYNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| SEQ ID | EIVLTQSPGTLSLSPGERATLSCRASQSVSSSYLAWYQQKPGQAPRLLIYGASSRATGI |
| NO: 12 | PDRFSGSGSGTDFTLTISRLEPEDFAVYYCQQYGSSPLTFGGGTKVEIKRTVAAPSVFI |
| | FPPSDEQLKSGTASVVCLLNNFYPREAKVQWKVDNALQSGNSQESVTEQDSKDSTYSLS |
| | STLTLSKADYEKHKVYACEVTHQGLSSPVTKSENRGEC |
| SEQ ID | QAQVVESGGGVVQSGRSLRLSCAASGFAFSSYGMHWVRQAPGKGLEWVAVIWYDGSNKY |
| NO: 13 | YADSVRGRFTISRDNSENTLYLQMNSLRAEDTAVYYCARDHYGSGVHHYFYYGLDVWGQ |
| | GTTVTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKRVEPKSCDKTHTCP |
| | PCPAPEFEGGPSVFLFPPKPKDTLMASRTPEVTCVVVDVSHEDPEVKENWYVDGVEVHN |
| | AKTKPREEQYNSTYRVVSVLTVLAQDWLNGKEYKCKVSNKALPASIEKTISKAKGQPRE |
| | PQVYTLPPSREEMTKNQVSLTCLVKGFYPSDIAVEWESNGQPENNYKTTPPVLDSDGSF |
| | FLYSKLTVDKSRWQQGNVFSCSVMHEALHNAYTQKSLSLSPGK |
| SEQ ID | QAQVVESGGGVVQSGRSLRLSCAASGFAFSSYGMHWVRQAPGKGLEWVAVIWYDGSNKY |
| NO: 14 | YADSVRGRFTISRDNSENTLYLQMNSLRAEDTAVYYCARDHYGSGVHHYFYYGLDVWGQ |
| | GTTVTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKRVEPKSCDKTHTCP |
| | PCPAPELLGGPSVFLFPPKPKDTLMISRTPEVTCVVVDVSHEDPEVKENWYVDGVEVHN |
| | AKTKPREEQYASTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| SEQ ID | QAQVVESGGGVVQSGRSLRLSCAASGFAFSSYGMHWVRQAPGKGLEWVAVIWYDGSNKY |
| NO: 15 | YADSVRGRFTISRDNSENTLYLQMNSLRAEDTAVYYCARDHYGSGVHHYFYYGLDVWGQ |
| | GTTVTVSSASTKGPSVFPLAPCSRSTSESTAALGCLVKDYFPEPVTVSWNSGALTSGVH |
| | TFPAVLQSSGLYSLSSVVTVPSSSLGTKTYTCNVDHKPSNTKVDKRVESKYGPPCPSCP |
| | APEFLGGPSVFLFPPKPKDTLMISRTPEVTCVVVDVSQEDPEVQFNWYVDGVEVHNAKT |
| | KPREEQFNSTYRVVSVLTVLHQDWLNGKEYKCKVSNKGLPSSIEKTISKAKGQPREPQV |
| | YTLPPSQEEMTKNQVSLTCLVKGFYPSDIAVEWESNGQPENNYKTTPPVLDSDGSFFLY |
| | SRLTVDKSRWQEGNVFSCSVMHEALHNHYTQKSLSLSLGK |
| SEQ ID | QAQVVESGGGVVQSGRSLRLSCAASGFAFSSYGMHWVRQAPGKGLEWVAVIWYDGSNKY |
| NO: 16 | YADSVRGRFTISRDNSENTLYLQMNSLRAEDTAVYYCARDHYGSGVHHYFYYGLDVWGQ |
| | GTTVTVSSASTKGPSVFPLAPCSRSTSESTAALGCLVKDYFPEPVTVSWNSGALTSGVH |
| | TFPAVLQSSGLYSLSSVVTVPSSSLGTKTYTCNVDHKPSNTKVDKRVESKYGPPCPPCP |
| | APEFLGGPSVFLFPPKPKDTLMISRTPEVTCVVVDVSQEDPEVQENWYVDGVEVHNAKT |
| | KPREEQFNSTYRVVSVLTVLHQDWLNGKEYKCKVSNKGLPSSIEKTISKAKGQPREPQV |
| | YTLPPSQEEMTKNQVSLTCLVKGFYPSDIAVEWESNGQPENNYKTTPPVLDSDGSFFLY |
| | SRLTVDKSRWQEGNVFSCSVMHEALHNHYTQKSLSLSLGK |
| SEQ ID | QAQVVESGGGVVQSGRSLRLSCAASGFAFSSYGMHWVRQAPGKGLEWVAVIWYDGSNKY |
| NO: 17 | YADSVRGRFTISRDNSENTLYLQMNSLRAEDTAVYYCARDHYGSGVHHYFYYGLDVWGQ |
| | GTTVTVSSASTKGPSVFPLAPCSRSTSESTAALGCLVKDYFPEPVTVSWNSGALTSGVH |
| | TFPAVLQSSGLYSLSSVVTVPSSSLGTKTYTCNVDHKPSNTKVDKRVESKYGPPCPPCP |
| | APEFEGGPSVFLFPPKPKDTLMISRTPEVTCVVVDVSQEDPEVQFNWYVDGVEVHNAKT |
| | KPREEQFNSTYRVVSVLTVLHQDWLNGKEYKCKVSNKGLPSSIEKTISKAKGQPREPQV |
| | YTLPPSQEEMTKNQVSLTCLVKGFYPSDIAVEWESNGQPENNYKTTPPVLDSDGSFFLY |
| | SRLTVDKSRWQEGNVFSCSVMHEALHNHYTQKSLSLSLGK |
| SEQ ID | QAQVVESGGGVVQSGRSLRLSCAASGFAFSSYGMHWVRQAPGKGLEWVAVIWYDGSNKY |
| NO: 18 | YADSVRGRFTISRDNSENTLYLQMNSLRAEDTAVYYCARDHYGSGVHHYFYYGLDVWGQ |
| | GTTVTVSSASTKGPSVFPLAPCSRSTSESTAALGCLVKDYFPEPVTVSWNSGALTSGVH |
| | TFPAVLQSSGLYSLSSVVTVPSSNFGTQTYTCNVDHKPSNTKVDKTVERKCCVECPPCP |
| | APPVAGPSVFLFPPKPKDTLMISRTPEVTCVVVDVSHEDPEVQENWYVDGVEVHNAKTK |
| | PREEQFNSTFRVVSVLTVVHQDWLNGKEYKCKVSNKGLPAPIEKTISKTKGQPREPQVY |
| | TLPPSREEMTKNQVSLTCLVKGFYPSDIAVEWESNGQPENNYKTTPPMLDSDGSFFLYS |
| | KLTVDKSRWQQGNVFSCSVMHEALHNHYTQKSLSLSPGK |
| SEQ ID | QAQVVESGGGVVQSGRSLRLSCAASGFAFSSYGMHWVRQAPGKGLEWVAVIWYDGSNKY |
| NO: 19 | YADSVRGRFTISRDNSENTLYLQMNSLRAEDTAVYYCARDHYGSGVHHYFYYGLDVWGQ |
| | GTTVTVSSASTKGPSVFPLAPCSRSTSESTAALGCLVKDYFPEPVTVSWNSGALTSGVH |
| | TFPAVLQSSGLYSLSSVVTVPSSNFGTQTYTCNVDHKPSNTKVDKTVERKCCVECPPCP |
| | APPVAGPSVFLFPPKPKDTLMISRTPEVTCVVVDVSQEDPEVQFNWYVDGVEVHNAKTK |
| | PREEQFNSTYRVVSVLTVLHQDWLNGKEYKCKVSNKGLPSSIEKTISKAKGQPREPQVY |
| | TLPPSQEEMTKNQVSLTCLVKGFYPSDIAVEWESNGQPENNYKTTPPVLDSDGSFFLYS |
| | RLTVDKSRWQEGNVFSCSVMHEALHNHYTQKSLSLSLGK |

### Statements

1. A human variant IgG Fc domain, having a mutation or combination of mutations with respect to the parent polypeptide sequence which reduce binding of the Fc domain to any FcγR, and a mutation or combination of mutations with respect to the parent polypeptide sequence which reduce binding of the Fc domain to FcRn, whereby maternofetal transfer is inhibited with respect to the parent sequence, preferably by at least 95% or more.
2. An isolated polypeptide comprising a human variant IgG Fc domain according to statement 1, the Fc domain having a mutation or combination of mutations with respect to the parent sequence which reduce binding of the Fc domain to any FcγR, and a mutation or combination of mutations with respect to the parent sequence which reduce binding of the Fc domain to FcRn, whereby maternofetal transfer is inhibited with respect to the parent sequence, preferably by at least 95% or more.
3. An isolated polypeptide according to statement 2 comprising a human variant IgG Fc domain comprising amino acid substitutions numbered according to the Kabat EU index numbering systern relative to a human wild-type Fc domain, wherein:
   a) (i) positions 234 and 235 are each substituted with alanine or
      (ii) positions 234 and 235 are each substituted with alanine and position 331 is substituted with serine or
      (iii) position 234 is substituted with phenylalanine, position 235 is substituted with glutamic acid and position 331 is substituted with serine or
      (iv) position 328 is substituted with arginine and arginine is inserted after position 236;
   and
   b) (i) position 253 is substituted with alanine or
      (ii) position 435 is substituted with alanine or
      (iii) positions 235, 310 and 435 are substituted with alanine or
      (iv) position 310 is substituted with alanine and position 435 is substituted with glutamine
   wherein said polypeptide has reduced binding to at least one Fe gamma receptor (FcγR) and to FcRn, when compared to the unsubstituted polypeptide comprising the parent Fc domain.
4. The polypeptide of any one of statements 2 or 3, or the variant IgG Fc domain of statement 1 where the polypeptide forms a part of an antibody or fragment thereof and which exhibits an increase, such as of at least 10 fold, in the concentration of antibody or antibody fragment, respectively, needed to give 50% binding, or activation, in a binding or cellular activation assay and/or a decrease in the Km binding constant, such as by a factor of at least 10 fold, by SPR or equivalent method, to at least one Fcγ receptor and to FcRn when compared to the same polypeptide comprising the parent Fc domain.
5. The polypeptide of any one of statement 2, 3 and 4, or the variant IgG Fc domain of statement 1 or 4 wherein at least one FcγR is selected from FcγRi, FcγRIIa, FcγRIIb, FcγIIIa, and FcγRIIIb, preferably FcγIIb and FcγIIIa.
6. The polypeptide or variant IgG Fc domain of any of the preceding statements, wherein the IgG Fc domain is selected from the group consisting of human immunoglobulin G class 1 (IgG1) Fc domain, human immunoglobulin G class 2 (IgG2) Fc domain, human immunoglobulin G class 3 (IgG3) Fc domain, and human immunoglobulin G class 4 (IgG4) Fc domain, preferably human immunoglobulin G class 1 (IgG1) Fc domain.
7. The polypeptide or variant IgG Fc domain of any of the preceding statements, wherein the parent polypeptide sequence is a wild type sequence, as appropriate for the IgG subclass of the Fc domain, or is a modified wild type sequence comprising any of the following mutations: IgG1(N297A), IgG1(N297G), IgG1(L234F/L235E/P331S), IgG1(L234A/L235A), IgG1(L235V/F243L/R292P/Y300L/P396L), IgG1(L234F/L235E/P331S), IgG1(L234A/L235A/P329G), IgG1(S354C/T366W), IgG1(Y349C/T366S/L368A/Y407V), IgG1(M252Y/S254T/T256E), IgG1/2(S239D/I332E), IgG2(C131S/R133K/C219S), IgG2(H268Q/R355Q/Q419E/N434A), IgG2/4(M428L/N434S), IgG4(S228P), IgG4(S228P/F234A/L235A), IgG4(S228P/L235E), IgG4(S241P/F234A/L235A), IgG4(F405L/R409K).
8. The polypeptide or variant IgG Fc domain of any of the preceding statements, comprising an amino acid sequence that is at least 80%, preferably at least 90% identical to the amino acid sequence selected from the group consisting of SEQ ID NO:6 to SEQ ID NO:9.
9. The polypeptide or variant IgG Fc domain of any of the preceding statements, wherein the polypeptide further comprises an antigen binding domain, such as, an antigen-binding domain of a monoclonal antibody or an antigen-binding fragment thereof.
10. The polypeptide of any of statements 2-8, comprising an amino acid sequence that is at least 80%, preferably at least 90%, more preferably at least 95% identical to the amino acid sequence of a heavy chain of SEQ ID NO:13, such as the polypeptide of SEQ ID NO 13, optionally together with an antibody light chain of SEQ ID NO:12.
11. An antibody comprising a heavy chain having a sequence combination of SEQ ID NO:10 as the variable heavy chain sequence and SEQ ID NO 5 as the heavy chain constant region and a light chain having a sequence of SEQ ID no 12, further comprising the substitutions of statement 3.
12. A conjugate comprising a variant IgG Fc domain, polypeptide or antibody according to any preceding statement and a therapeutic moiety.
13. A nucleic acid comprising a nucleotide sequence encoding the variant IgG Fc domain or polypeptide according to any of statements 1 to 9, antibody of statement 10 or conjugate of statement 11.
14. A vector comprising the nucleic acid of statement 13.
15. A host cell comprising the nucleic acid according to statement 13 or 14
16. A pharmaceutical composition comprising a variant IgG Fc domain of statement 1, the polypeptide according to any one of statements 2 to 10, an antibody according to statement 11, a conjugate according to statement 12 a nucleic acid of statement 13, or the vector of statement 14, and a pharmaceutically acceptable excipient.
17. A variant IgG Fc domain of statement 1, polypeptide according to any one of statements 2 to 9, an antibody according to statement 11, a conjugate according to statement 12, a nucleic acid of statement 13, or the vector of statement 14, or a pharmaceutical composition according to statement 16 for use in therapy of a mammal, preferably a human, more preferably a woman, such as a woman of reproductive age, for example 15-45 years old, even more preferably a pregnant woman.
18. A method to reduce binding to at least one Fcγ receptor and to FcRn in a parent polypeptide comprising an Fc domain wherein:
   a) (i) positions 234 and 235 are each substituted with alanine or
      (ii) positions 234 and 235 are each substituted with alanine and position 331 is substituted with serine or
      (iii) position 234 is substituted with phenylalanine, position 235 is substituted with glutamic acid and position 331 is substituted with serine or
      (iv) position 328 is substituted with arginine and arginine is inserted after position 236;
   and
   b) (i) position 253 is substituted with alanine or
      (ii) position 435 is substituted with alanine or
      (iii) positions 235, 310 and 435 are substituted with alanine or
      (iv) position 310 is substituted with alanine and position 435 is substituted with glutamine
optionally wherein the polypeptide containing the substitutions is expressed, suitably in the form of an antibody or fragment thereof,
and optionally combined with a pharmaceutically acceptable carrier or excipient.
19. A method of treating a mammal, preferably a human, more preferably a woman, such as a woman of reproductive age, for example 15-45 years old, even more preferably a pregnant woman, the method comprising administering an effective amount of any one or more of:
   (a) a variant IgG Fc domain or polypeptide of statements 1-10,
   (b) an antibody according to statement 11,
   (c) a conjugate according to statement 12,
   (d) a nucleic acid of statement 13,
   (e) a vector of statement 14,
   (f) a cell according to statement 15, or
   (g) a pharmaceutical composition according to statement 16.

1. An isolated polypeptide for use in medicine in a pregnant woman, wherein the polypeptide comprises a human variant IgG Fc domain having a mutation or combination of mutations with respect to the parent sequence which reduces binding of the Fc domain to any FcγR, and a mutation or combination of mutations with respect to the parent sequence which reduce binding of the Fc domain to FcRn, whereby maternofetal transfer is inhibited with respect to the parent sequence by at least 95% or more.

2. An isolated polypeptide for use in the treatment or prevention of a disorder of pregnancy in a pregnant woman, the polypeptide comprising a human variant IgG Fc domain having a mutation or combination of mutations with respect to the parent sequence which reduce binding of the Fc domain to any FcγR, and a mutation or combination of mutations with respect to the parent sequence which reduce binding of the Fc domain to FcRn, whereby maternofetal transfer is inhibited with respect to the parent sequence by at least 95% or more.

3. A method of treating a pregnant woman, the method comprising administering an effective amount of an isolated polypeptide, the polypeptide comprising a human variant IgG Fc domain having a mutation or combination of mutations with respect to the parent sequence which reduce binding of the Fc domain to any FcγR, and a mutation or combination of mutations with respect to the parent sequence which reduce binding of the Fc domain to FcRn, whereby maternofetal transfer is inhibited with respect to the parent sequence by at least 95% or more.

4. An isolated polypeptide for use in medicine in a pregnant woman according to claim 1, or for use in therapy of a pregnant woman according to claim 2, or a method of treatment of a pregnant woman according to claim 3, wherein the polypeptide comprises a human variant IgG Fc domain comprising amino acid substitutions numbered according to the Kabat EU index numbering system relative to a human wild-type Fc domain, wherein:
a) (i) positions 234 and 235 are each substituted with alanine or
(ii) positions 234 and 235 are each substituted with alanine and position 331 is substituted with serine or
(iii) position 234 is substituted with phenylalanine, position 235 is substituted with glutamic acid and position 331 is substituted with serine or
(iv) position 328 is substituted with arginine and arginine is inserted after position 236;
and
b) (i) position 253 is substituted with alanine or
(ii) position 435 is substituted with alanine or
(iii) positions 235, 310 and 435 are substituted with alanine or
(iv) position 310 is substituted with alanine and position 435 is substituted with glutamine
wherein said polypeptide has reduced binding to at least one Fc gamma receptor (FcγR) and to FcRn, when compared to the unsubstituted polypeptide comprising the parent Fc domain.

5. An isolated polypeptide for use in medicine in a pregnant woman according to claim 1 or 4, or for use in therapy of a pregnant woman according to claim 2 or 4, or a method of treatment of a pregnant woman according to claim 3 or 4, wherein the polypeptide forms a part of an antibody or fragment thereof and the polypeptide exhibits an increase in the concentration of antibody or antibody fragment, respectively, needed to give 50% binding, or activation, in a binding or cellular activation assay and/or a decrease in the Km binding constant, such as by a factor of at least 10 fold, by SPR or equivalent method, to at least one Fcγ receptor and to FcRn when compared to the same polypeptide comprising the parent Fc domain.

6. An isolated polypeptide for use in medicine in a pregnant woman according to any one of claim 1 or 4 or 5, or for use in therapy of a pregnant woman according to any one of claim 2 or 4 or 5, or a method of treatment of a pregnant woman according to any one of claim 3 or 4 or 5, wherein at least one FcγR is selected from FcγRi, FcγRIIa, FcγRIIb, FcγIIIa, and FcγRIIIb, preferably FcγIIb and FcγIIIa.

7. An isolated polypeptide for use in medicine in a pregnant woman according to any one of claims 1 or 4-6, or for use in therapy of a pregnant woman according to any one of claims 2 or 4-6, or a method of treatment of a pregnant woman according to any one of claim 3-6, wherein the IgG Fc domain is selected from the group consisting of human immunoglobulin G class 1 (IgG1) Fc domain, human immunoglobulin G class 2 (IgG2) Fc domain, human immunoglobulin G class 3 (IgG3) Fc domain, and human immunoglobulin G class 4 (IgG4) Fc domain, preferably human immunoglobulin G class 1 (IgG1) Fc domain.

8. An isolated polypeptide for use in medicine in a pregnant woman according to any one of claims 1 or 4-7, or for use in therapy of a pregnant woman according to any one of claims 2 or 4-7, or a method of treatment of a pregnant woman according to any one of claim 3 7, wherein the parent polypeptide sequence is a wild type sequence, as appropriate for the IgG subclass of the Fc domain, or is a modified wild type sequence comprising any of the following mutations: IgG1(N297A), IgG1(N297G), IgG1(L234F/L235E/P331S), IgG1(L234A/L235A), IgG1(L235V/F243L/R292P/Y300L/P396L), IgG1(L234F/L235E/P331S), IgG1(L234A/L235A/P329G), IgG1(S354C/T366W), IgG1(Y349C/T366S/L368A/Y407V), IgG1(M252Y/S254T/T256E), IgG1/2(S239D/I332E), IgG2(C131S/R133K/C219S), IgG2(H268Q/R355Q/Q419E/N434A), IgG2/4(M428L/N434S), IgG4(S228P), IgG4(S228P/F234A/L235A), IgG4(S228P/L235E), IgG4(S241P/F234A/L235A), IgG4(F405L/R409K).

9. An isolated polypeptide for use in medicine in a pregnant woman according to any one of claims 1 or 4-8, or for use in therapy of a pregnant woman according to any one of claims 2 or 4-8, or a method of treatment of a pregnant woman according to any one of claim 3-8, wherein the polypeptide comprises an amino acid sequence that is at least 80%, preferably at least 90% identical to the amino acid sequence selected from the group consisting of SEQ ID NO:6 to SEQ ID NO:9.

10. An isolated polypeptide for use in medicine in a pregnant woman according to any one of claims 1 or 4-9, or for use in therapy of a pregnant woman according to any one of claims 2 or 4-9, or a method of treatment of a pregnant woman according to any one of claim 3-9, wherein the polypeptide further comprises an antigen binding domain, such as, an antigen-binding domain of a monoclonal antibody or an antigen-binding fragment thereof.

11. An isolated polypeptide for use in medicine in a pregnant woman according to any one of claims 1 or 4-10, or for use in therapy of a pregnant woman according to any one of claims 2 or 4-10, or a method of treatment of a pregnant woman according to any one of claim 3-10, wherein the polypeptide comprises an amino acid sequence that is at least 80%, preferably at least 90%, more preferably at least 95% identical to the amino acid sequence of a heavy chain of SEQ ID NO:13, such as the polypeptide of SEQ ID NO 13, optionally together with an antibody light chain of SEQ ID NO:12.

12. An isolated polypeptide for use in medicine in a pregnant woman according to any one of claims 1 or 4-11, or for use in therapy of a pregnant woman according to any one of claims 2 or 4-11, or a method of treatment of a pregnant woman according to any one of claim 3-11, wherein the polypeptide is an antibody comprising a heavy chain having a sequence combination of SEQ ID NO:10 as the variable heavy chain sequence and SEQ ID NO 5 as the heavy chain constant region and a light chain having a sequence of SEQ ID no 12, further comprising any of the substitutions of claim 4.

13. An isolated polypeptide for use in medicine in a pregnant woman according to any one of claims 1 or 4-12, or for use in therapy of a pregnant woman according to any one of claims 2 or 4-12, or a method of treatment of a pregnant woman according to any one of claim 3-12, wherein the polypeptide is in the form of a conjugate comprising a variant IgG Fc domain, polypeptide or antibody according to any preceding claim and a therapeutic moiety.

14. A nucleic acid comprising a nucleotide sequence encoding a polypeptide as disclosed in any one of claims 1-13 for use in therapy of a pregnant woman, for example in the treatment or prevention of a disorder of pregnancy.

15. A vector comprising the nucleic acid of claim 15 for use in therapy of a pregnant woman, for example in the treatment or prevention of a disorder of pregnancy.

16. A host cell comprising the nucleic acid according to claim 14 or 15 for use in therapy of a pregnant woman, for example in the treatment or prevention of a disorder of pregnancy.

17. An isolated polypeptide for use in medicine in a pregnant woman according to any one of claims 1 or 4-12, or for use in therapy of a pregnant woman according to any one of claims 2 or 4-12, or a method of treatment of a pregnant woman according to any one of claim 3-12, or a nucleic acid for use according to claim 14, vector for use according to claim 15 or cell for use according to claim 16 wherein the polypeptide is in the form of a pharmaceutical composition which comprises a pharmaceutically acceptable excipient.

18. An isolated polypeptide for use, method of treatment, nucleic acid for use, vector for use or cell for use according to any one of claim 1-17, in the treatment or prevention of a disorder of pregnancy,
optionally wherein the disorder or disease is a hypertension-related condition or pre-eclampsia/eclampsia, or
optionally wherein the disorder or disease is pre-eclampsia/eclampsia, and the polypeptide is an anti-VEGFR-1 antibody which inhibits binding of VEGF and/or PGF to sVEGFR-1, or
optionally wherein the disorder or disease is pre-eclampsia/eclampsia, and the polypeptide comprises or consists of the polypeptide of SEQ ID NO 13, provided together with an antibody light chain of SEQ ID NO:12, in the form of an antibody, optionally in the form of a pharmaceutically acceptable composition in combination with a pharmaceutically acceptable excipient.

19. An antibody comprising the polypeptide of SEQ ID NO 13 together with an antibody light chain of SEQ ID NO:12, optionally formulated as a pharmaceutically acceptable composition with an excipient or carrier.
